(19) United States
(12) Patent Application Publication
(10) Pub. No.: US 2007/0042001 A1
(43) Pub. Date: Feb. 22, 2007

(54) Influenza recombinant subunit vaccine

(75) Inventors: Carolyn L. Weeks-Levy, Honolulu, HI (US); David Edward Clements, Honolulu, HI (US); Steven A. Ogata, Kailua, HI (US)

Correspondence Address: Paradise Patent Services, Inc.;Attn: George Darby, P.O. Box 893010, Mililani, HI, 96789-0010, US

(73) Assignee: HAWAII BIOTECH, INC.

(21) Appl. No.: 11/505,694

(22) Filed: Aug. 16, 2006

Related U.S. Application Data
(60) Provisional application No. 60/708,988, filed on Aug. 16, 2005.

(51) Int. Cl. A61K 39/145; C07H 21/04; C12N 7/00; C12N 5/06; C07K 14/11

(52) U.S. Cl. 424/209.1; 435/235.1; 435/69.3; 435/348; 435/456; 530/350; 536/23.72

(57) ABSTRACT

The invention provides influenza proteins, including subunit proteins and immunogenic compositions that can be utilized, with or without adjuvants, as vaccines to protect against influenza infection in animal models and humans. The recombinant proteins are expressed from transformed insect cells that contain integrated copies of the appropriate expression cassettes in their genome. The invention uses a Drosophila melanogaster expression system to provide high yields of recombinant subunit proteins with native-like conformation.

### RELATED APPLICATION

This application claims the benefit of U.S. Provisional Patent Application No. 60/708,988, filed Aug. 16, 2005, the disclosures and drawings of which prior application are hereby incorporated by reference in their entirety.

### INCORPORATION OF SEQUENCE LISTING

A sequence listing file in ST.25 format on CD-ROM is appended to this application and fully incorporated herein by reference. The sequence listing information recorded in computer readable form is identical to the written sequence listing (per WIPO ST.25 para. 39, the information recorded on the form is identical to the written sequence listing). With respect to the appended CD-ROMs, the format is ISO 9660; the operating system compatibility is MS-Windows; the single file contained on each CD-ROM is named “FLU.S2.ADJ.03.ST25.txt” and is a text file produced by PatentIn 3.3 software; the file size in bytes is 22 KB; and the date of file creation is 15 Aug. 2006. The contents of the two CD-ROMs submitted herewith are identical.

### BACKGROUND OF THE INVENTION

1. Technical Field

The invention relates to vaccine formulations designed to protect against influenza. In particular, the vaccine formulations comprise recombinant subunit proteins derived from influenza virus, and optionally include one or more adjuvants. “Subunit protein” is defined here as any protein derived or expressed independently from the complete organism that it is derived from. Furthermore, a subunit protein may represent a full length native protein sequence or any fraction of the full length native protein sequence. Additionally, a subunit protein may contain in addition to the full length or partial protein sequence, one or more sequences, which may contain sequences that are homologous or heterologous to the organism from which the primary sequence was derived. This definition is significantly broader than the concept of a subunit protein as a single protein molecule that co-assembles with other protein molecules to form a multimeric or oligomeric protein. The subunit proteins of the invention are produced in a cellular production system by means of recombinant DNA methods and, after purification, are formulated in a vaccine.

2. Related Art

Each year an estimated 20% of the US population will develop influenza. Approximately 150,000 of those infected will be hospitalized (Schoenbaum, Am, J, Med. (1987) 82(Suppl 6A):26-30; Simonsen et al., Arch. Intern. Med. (1998) 158:1923-1928). On average, 36,000 deaths per year can be anticipated from this disease (Simonsen et al., Am. J. Pub. Health (1997) 87:1944-1950) with deaths climbing to 100,000 during pandemic years (Ghendon, World Health Stat Q (1992) 45:306.). In 1918, the most deadly pandemic in the last 100 years killed over 500,000 people in the United States alone (Taubenberger, Avian Diseases (2003) 47 (Suppl 3):789-791). The elderly (>65 years) and the very young are most susceptible to complications from the influenza virus (CDC, MMWR, (2001) 50 (RR-04): 1-63; Neuzil et al., JAMA (1999) 281:901-907). The cost of the influenza disease burden in the United States during 1993 was estimated at $14.6 billion (Kennedy, Nurse Pract. (1998) 23:17-28).

Influenza virus is an orthomyxovirus containing eight single stranded RNA segments. The eight segments code for the following proteins: HA (hemagglutinin), NA (neuraminidase), M1 (matrix), M2 (transmembrane), NP (nucleoprotein), PB2 (polymerase), PB1 (polymerase), PA (polymerase), NEP (viral assembly), and NS1 (interferon antagonist) (Harper et al., Clin. Med. Lab. (2002) 22:863-882; Hilleman, Vaccine (2002) 20:3068-3087; Cox et al., Scandanavian J. of Immun. (2003) 59:1-15). The most abundant protein on the virus surface is HA protein. The HA protein is responsible for attachment of the virus to the sialic acid-containing receptors on the host cell surface and fusion of the viral and endosome membranes for release of the viral ribonucleotide NP (RNPs) complexes into the cytoplasm of the host cell (Cox et al., Scandanavian J. of Immun. (2003) 59:1-15). NA is also on the surface but in lower copy number than HA. NA protein cleaves sialic acid and plays an important role in viral entry and release. The M2 protein is also present on the surface (24 amino acids of the 97 amino acid protein) of the virus but is in much less abundance than HA or NA.

There are three types of influenza virus, A, B and C (types are based on the sequence of NP and M1 proteins). Influenza type C causes a mild respiratory illness and is not included in current flu vaccine formulations. Type B virus circulates widely among humans and is included in the current flu formulations produced each year. Type B viruses have no subtypes as they contain only one type of HA and NA proteins. On the other hand, type A viruses contain various types of HA and NA proteins that vary in sequence and, as a result, type A viruses are designated as subtypes based on the make up of these two proteins. For the type A viruses there are 16 HA subtypes and 9 NA subtypes. Only 5 of the 16 HA subtypes and 2 of the 9 NA subtypes are infectious in humans; H1, H2, H3, H5, H9 and N1, N2, respectively (Cox et al., Scandanavian J. of Immun. (2003) 59:1-15).

The hemagglutinin (HA) protein of the influenza virus is the most abundant protein on the surface of the virus and is primarily responsible for the humoral immune response against the virus upon infection. Therefore, HA is the leading candidate for inclusion in a subunit vaccine for influenza. While the antibody responses directed against the surface protein, HA, is a key component in a protective immune response, cellular immune responses directed against various structural and nonstructural proteins of the influenza virus are thought to also contribute to protection.

A clinical trial was conducted in 63 volunteers to evaluate the importance of cytotoxic T-cell immunity in protection of infected individuals against influenza disease (McMichael et al., N. Engl. J. Med. (1983) 309:13-17). The volunteers were infected with influenza at the Medical Research Council Common Cold Unit in Salisbury, UK, and were quarantined and evaluated for 10 days. Cytotoxic T-cell activity of the volunteers was measured during the 10 day evaluation period. The authors conclude that data obtained during the study supports the hypothesis that cytotoxic T-cell lymphocytes play a part in recovery from influenza infection and that vaccines with the potential to stimulate more prolonged T-cell immunity might prove useful.

Cellular immunity has been well established as a key mechanism in virus clearance in the murine model (Karzon D T, Semin Virol. (1996) 7:265-271). Proteins internal to the virus such as the M1 protein may be useful for the purpose of eliciting cellular immune responses. Using HA protein and the internal influenza proteins, with or without the use of appropriate adjuvants, an immune response directed at both the humoral and cellular level can be achieved.

As mentioned previously, influenza HA protein is the primary protein found on the surface of the virus. The HA found on the surface of the viron is in a trimeric form. The trimer is anchored to the viral membrane by transmembrane spanning sequences at the carboxy-terminal end of each of the three monomers. The main protective efficacy of influenza vaccine is attributed to anti-hemagglutinin antibodies stimulated by HA protein; the anti-HA antibodies inhibit the attachment of the virus to cells (Virelizier J L, J. Immunol. (1975) 115:434-439). Inhibition of virus attachment protects individuals against infection or serious illness depending on the magnitude of anti-hemagglutinin titers stimulated by vaccination. The fusion of influenza virus to the host cell depends on the structure of the HA molecule. During maturation of the virus during the replication cycle, the HA protein is cleaved immediately N-terminal to the fusion peptide. This cleavage of HA0 to HA1 and HA2 is essential for fusion to occur (Steinhauer D A, Virology (1999) 258:1-20). Another necessary step in the fusion process requires that HA trimerizes (Danieli et al., J. Cell Biol. (1996) 133:559-569). Therefore, inhibition of this viral process is very dependent on proper conformation epitopes of the HA molecule and trimers thereof, and binding of paratopes to those epitopes. This highlights the importance of raising an immune response to conformationally relevant HA protein.

While HA is the primary protein in existing influenza vaccine formulations and influenza vaccines under development, the use of this protein in vaccines is confounded by the nature of HA in type A influenza viruses which are of the greatest concern. Type A viruses undergo “antigenic drift” over time as the sequence in HA under goes small changes, resulting in the need to substitute “newer” strains of influenza virus in the vaccine each year to keep up with the changes in the current circulating strains (the U.S. Food and Drug Administration (“FDA”) recommends strains each year to be included in influenza vaccine for administration in the U.S.). Strains that drift from each other contain common antigenic properties and therefore maintain the same HA subtype, however, the changes are significant enough to result in differences in antigenic properties. As a result, the FDA recommends virus strains to be included in a current year's vaccine along with alternate strains to keep in line with HA drift to afford the maximum protection following immunization. More substantial changes in the make up of type A viruses that result from recombinations of circulating strains are referred to as “antigenic shift”. These shifts are primarily in the HA gene and result in new strains being formed. As there is no pre-existing immunity to these new strains, they are often associated with pandemics of influenza (Nicholson et al., Lancet (2003) 362:1733-1745). The existence of both antigenic shift and drift pose significant challenges in preparing influenza vaccines with existing vaccine technology and for any new technology designed to produce improved influenza vaccines.

Influenza vaccines marketed in the United States are currently produced in embryonated chicken eggs. The inactivated vaccines contain primarily hemagglutinin (“HA”) protein after inactivation of live virus and purification of viral protein. HA binds to a sialic acid residue on the cell to be infected. The name of HA derives from the protein's ability to adhere to red blood cells and cause them to agglutinate, or clump together. Inactivation of the virus is accomplished through the use of agents such as formalin, which is a compound that is known to cross-link protein and damage epitopes. Influenza production procedures (use of embryonated chicken eggs) inherently limit the amount of influenza vaccine that can be produced prior to each year's flu season. In addition, impurities in the inactivated vaccines and preservatives added to the vaccines can lead to adverse events in those immunized with these vaccines.

In general, inactivated “split” (purified virus disrupted with chemicals such as Tween 80 to solubilize the envelope of the virus) influenza vaccine formulations are well tolerated in human subjects; mild soreness at the site of injection is the most common complaint (Margolis et al., JAMA (1990) 264:1139-1141; Nichol et al., Arch. Intern. Med. (1996) 156:1546-1550). Manufacturers of inactivated influenza vaccines do warn individuals with allergies to eggs to avoid vaccination with the product, however, immediate hypersensitivity reactions seem to be low (James et al., J. Pediatr. (1998) 133:624-628). Inactivated influenza vaccines have very rarely been associated with severe undesired side effects. Guillain-Barré syndrome has been associated with influenza vaccination at a rate of one per million vaccinees (Lasky et al., N. Engl. J. Med. (1998) 339:1797-1802).

Inactivated influenza vaccines are 60 to 100% effective in preventing morbidity and mortality, however, lower rates of efficacy are observed in the young and elderly. In addition, reduced efficacy in the general public occurs in years of poor antigenic match of the vaccine strain to the circulating strain (Beyer et al., Vaccine (2002) 20:1340-1353).

Suppression or impairment of either the humoral or cell mediated branch of the immune system can lead to increased susceptibility or severity of disease induced by infectious agents (e.g., opportunistic infections). In “immunosuppressed” individuals, the immune response is prevented or diminished (e.g., by administration of radiation, antimetabolites, antilymphocyte serum, or specific antibody). “Immunocompromised” or “immunodeficient” individuals have their immune system attenuated (e.g., by malnutrition, irradiation, cytotoxic chemotherapy, or diseases such as cancer or AIDS, or by primary immune deficiencies). Recent advances in understanding of aging and immunology have suggested that elderly subjects also show a decreased immunoresponsiveness, sometimes referred to as immunosenescence (Pawelec, Biogerontology (2003) 4:167-70; Mishto et al., Ageing Res. Rev. (2003) 2:419-32; McElhaney, Conn. Med. (2003) 67:469-74; Pawelec et al., Front. Biosci. (2002) 7:d1056-183; Katz et al., Immunol. Res. (2004) 29:113-24). Elderly and infant subjects (especially, non-suckling infants) are also recognized to be more susceptible to infectious diseases (e.g., influenza infection—Katz et al., supra) consistent with an impaired or immature immune system. Immunosuppressed, immunocompromised, immunosenescent, and non-suckling infant populations (collectively, the “immunodeficient population”) are at particular risk for many infectious diseases, but concomitantly are too vulnerable to the effects of reversion or mutation of attenuated live virus vaccines, and therefore are an important target audience for vaccine development. However, the fact that members of the immunodeficient population have some degree of immune impairment makes the challenge of developing an immunogenic and protective vaccine for the immunodeficient population particularly difficult.

The manufacturing process for influenza vaccine inherently limits the amount of vaccine that can be made in time for the upcoming flu season. The two major suppliers of flu vaccine for the United States are Aventis (Fluzone®) and Chiron (Fluvirin®). Both companies produce influenza virus in embryonated chicken eggs (90 million of them used per year for manufacture). The virus is harvested, inactivated (formaldehyde, and betapropiolactone, respectively), filtered, and purified by continuous zonal centrifugation. The resultant product is standardized by the HA content and contains 15 μg of each HA antigen subtype. Various other flu proteins are also contained in the vaccine in lower and various amounts. Inactivation steps tend to damage antigen epitopes, which in turn requires the use of more protein to provide an adequate immune response. The current inactivated vaccine formulations are not adjuvanted.

Manufacture of inactivated-virus vaccines for pandemic influenza strains is further complicated by the need to grow the virus strains under BSL-3 level conditions. In addition, avian strains of influenza are lethal to chicken embryos, necessitating the construction of suitable strains using reverse genetics that can be used for manufacture in embryonated chicken eggs (Wood, Vaccine (2002) 20:B40-B44).

For influenza vaccines, protective immunity is considered to be achieved if an individual mounts an anti-hemagglutinin titer of ≧1:40 and seroconversion to the influenza immunizing strain is considered to occur if a four-fold increase in titer is achieved. The level of anti-NA antibodies necessary to limit viral spread has not yet been defined (Ada and Jones, Curr. Topics Microbiol. Immunol. (1986) 128:1-54; Aymard-Henry et. al., Bull WHO (1973) 48:199-202; Beran et. al., Centr. Eur. J. Pub. Health (1998) 4:269-273; Bridges et al., JAMA (2000) 284:1655-1663 and Brydak, Influenza and its Prophylaxis (1998) 1^{st} ed. Springer P W N, Warsaw). Protein Sciences (Meriden, Conn.) produces baculovirus-expressed HA and NA influenza proteins. These proteins have been tested in animal models and in human clinical trials and have met with limited success (discussed below).

Protein Sciences has not licensed an influenza vaccine using these proteins. The baculovirus expression system (“BES”) has a number of biological and purification process limitations (Farrell et al., Biotech and Bioeng. (1998) 60(6):656-663). One major manufacturing challenge is that insect cells are infected with baculovirus carrying the gene to be expressed, leading to cell lysis during the infection. This process provides a challenge for purification as insect cell proteins are co-purified with the expressed protein and cellular enzymes are released that can degrade the desired protein products.

MedImmune's FluMist® is a newly licensed live attenuated vaccine that is administered by nasal spray to patients between the ages of 5 and 49. This new vaccine is not licensed for use in “at-risk” populations. MedImmune produced approximately 4 million doses of FluMist® vaccine for the 2003 flu season. This vaccine is also grown on embryonated chicken eggs. This vaccine is a live attenuated formulation that is delivered by nasal spray. Besides limitations in the amount of doses that can be manufactured each year, the vaccine is not licensed for use in the young and elderly populations, which need protection from influenza the most.

Antiviral compounds are available for combating influenza infections; however, they come with limitations on their use (Williams et al., Kaohsiung J. Med. Sci (2002) 18:421-434). Amantadine and rimantadine are effective for the prevention and treatment of influenza infection; however, they are only effective for type A viruses. Drug resistant virus strains have also been isolated from individuals treated with these compounds (Englund et al., Clin. Infec. Dis. (1998) 26:1418-1424). These drugs also have undesirable side effects (Dolin et al., N. Engl. J. Med. (1982) 307:580-584). Newer antiviral agents such as zanamivir (nasal spray) and oseltamivir (oral) block (by transition-state analog inhibition) influenza A and B enzyme NA. These drugs can prevent disease if given prophylactically and can lessen the duration of symptoms if given within 48 hours of infection. Zanamivir and oseltamivir have fewer side effects but are more expensive than amantadine and rimantadine. Oseltamivir (trade name, Tamiflu®) is marketed by Roche Holding AG, who is building a new production plant devoted to production of oseltamivir. Demand for oseltamivir is driven in part by fear of pandemic flu and the stockpiling of flu therapeutic drugs by governments, e.g., the U.K. It would, of course, be preferable to reduce the need for stockpiling flu therapies by immunizing populations.

The current methods for the production of influenza vaccine clearly are limited in meeting the increasing demand for a higher number of doses per year and for addressing needed improvements in the immunogenicity and efficacy in certain segments of the population. As a result, there is a clear need for improved technologies for influenza vaccine manufacture that will provide for increased numbers of doses of influenza vaccine that can be manufactured swiftly and without the need for BSL-3 level containment or embryonated chicken eggs. Improvements in the immunogenicity and possibly cross-protectiveness of the vaccine also need to be achieved to effectively provide vaccines in response to the seasonal epidemics and for potential pandemics.

In an effort to alleviate the short comings of the currently manufactured influenza vaccines, several alternative approaches to producing vaccines are currently being developed. The use of cell culture based systems is probably the most investigated of the areas being pursued. These systems are based on the use of alternative cell substrates to produce influenza vaccine virus strains in culture. The two main cell culture lines that are being tested are MDCK (Palache et al, Dev. Biol. Stand. (1999) 98:115-125) and Vero (Halperin et al, Vaccine (2002) 20(7-8):1240-1247, and Nicolson, Vaccine (2005) 22:2943-2952). The process that is used to process the virus grown in these cells for use in vaccines is the same as that used with egg produced virus. Therefore, the virus is still inactivated with chemicals which have the potential to damage epitopes on the antigens. While the use of these cell culture methods avoids the use of embryonated eggs there are new regulatory hurdles (clearance of adventitious agents) along with the limitations of traditional produced egg vaccine due to the similarities in the process.

DNA vaccines encoding the HA and NP genes have been evaluated in mouse challenge models (Williams et al., Kaohsiung J. Med. Sci. (2002) 18:421-434; Kemble and Greenberg, Vaccine (2003) 21:1789-1795). Vaccination with DNA encoding the NP gene resulted in protection from challenge with a heterologous influenza strain (Montgomery et al., DNA Cell Biol. (1993) 12:777-783). Protection from homologous virus challenge was accomplished after vaccination with DNA encoding HA in mice. Antibody responses induced by vaccination with DNA resulted in long-lived titers in the mice (Ulmer et al., Science (1993) 259:1745-1749). Even though the results with DNA vaccination are quite encouraging, safety issues will continue to be a problem with this approach to vaccination.

DNA vaccines encoding the influenza HA, M2, and NP genes have been evaluated as alternative vaccines for influenza. This method is obviously not dependent on eggs or mammalian cell culture. Most studies have only presented encouraging results in mice (Montgomery et al., 1993; Ulmer et al., Science (1993) 259:1745-1749; and Williams et al., Kaohsiung J. Med. Sci. (2002) 18:421-434). Reports of promising results in larger animals are very hard to find. As an example a M2-NP DNA that worked well in mice appears to have exacerbated disease following challenge in a pig model (Heinen et al., J. Gen. Virol. (2002) 82(Pt 11):2697-2707). While the potential exists for a DNA vaccine for influenza, there are still the safety issues that will continue to be a problem with this approach to vaccination.

Recombinant subunit protein vaccines have been proposed as the solution for many different vaccines. This technology base has also been investigated for influenza vaccines. Systems based on E. coli, yeast, insect cells, and mammalian cells have been utilized. The development of recombinant subunit vaccines for influenza is an attractive option because the need to grow virus is eliminated. Numerous studies have been reported for testing of recombinant subunit vaccine candidates in animal models and only a few have been tested in human clinical trials. Two major problems have hampered the development of influenza recombinant proteins. They are inability to express native-like proteins and low expression levels. For example, HA, the primary component for influenza vaccines has proven to be a difficult protein to express as a recombinant. Expression in Pichia of a membrane anchorless HA molecule has been reported (Saelens et al., Eur. J. Biochem. (1999) 260(1):166-175). While the expressed HA protein had appropriate structure based on antibody binding and resulted in partial protection when used to immunize mice, the product was not completely uniform in nature. The N-terminus was variable due to variable processing and the glycosylation patterns where heterogeneous also. Despite statements that the Pichia expressed HA protein has potential as a vaccine candidate there is no indication that this effort has been carried on for testing in humans.

The baculovirus expression system (BES) has also investigated as a system for the production of recombinant influenza subunits. An early report on the expression of full length HA using BES resulted in HA being localized on the surface of the insect cells (Kuroda et al., EMBO J. (1986) 6:1359-1365). Further studies were reported on the expression of soluble HA from BES (Valandschoot et al., Arch Virol. (1996) 141:1715-1726). This report on soluble baculovirus expressed HA like the Pichia expressed HA determined that the protein had some native-like characteristics, but was mostly aggregated and did not provide any protection when tested in a mouse model. The recombinant baculovirus-expressed HA proteins under development by Protein Sciences Corporation (PSC Meriden, Conn.) represent the most advanced recombinant influenza vaccines to date. The HA expressed by PSC represents the full length molecule and results in the localization on the host insect cells. The HA is purified through a series of steps following extraction from the membrane. An H5 HA vaccine based on this methodology has been evaluated in human clinical trials (Treanor et al., Vaccine (2001) 19:1732-1737). One hundred forty seven healthy adults were randomly assigned to receive two intramuscular injections of either 25, 45 or 90 μg each, one dose of 90 μg followed by a dose of 10 μg, or two doses of placebo; doses given at intervals of 21, 28 or 42 days. The vaccine was not adjuvanted. The clinical trial demonstrated that a neutralizing antibody titer of ≧1:80 was achieved in some individuals receiving a single dose of 90 μg (23%) or two doses of 90 μg (52%). The authors of this paper concluded that the immunogenicity of the vaccine needs to be improved.

Production of virus-like particles (VLP) containing influenza proteins utilizing BES has been reported (Latham and Galarza, J. Virol. (2001) 75(13):6154-6165). This methodology is currently being pursued by Novavax (Malvern, Pa.). VLPs consisting of HA, NA and M1 proteins have been produced and are being developed for use as vaccines (Pushko et al., Vaccine (2005) 23(50):5751-5759). The VLPs exhibit functional characteristics of influenza virus and were shown to inhibit replication of influenza virus after challenge of vaccinated Balb/c mice. The use of VLPs for influenza vaccination appears promising; however, the authors do cite manufacturing issues that need to be solved in order to develop a scalable manufacturing process that could be used to meet production needs.

Despite the advancements in the development of recombinant influenza vaccines thus far, one key issue remaining is the ability to produce high quality immunogens that will increase the overall seroprotective immune response, especially in elderly and other sectors of the immunodeficient population. In addition, production systems must be developed that can produce enough vaccine doses, even on short notice, to cover the populations that need them.

It is important that a recombinant expression system be able to produce both a high quality product and high yields of the desired product. In an effort to meet these criteria, the Drosophila expression system, as defined below, was selected by the inventors for the expression of influenza recombinant subunit proteins. This system has been shown to be able to express heterologous proteins that maintain native-like biological structure and function (Bin et al, Biochem J. (1996) 313:57-64 and Incardona and Rosenberry, Mol. Biol. of the Cell (1996) 7:595-611). The Drosophila expression system is also capable of producing high yields of product. The use of an efficient recombinant expression system will ultimately lower the cost per dose of a vaccine and enhance the commercial potential of the product. To the inventors' knowledge, using the Drosophila expression system to produce influenza HA and M1 proteins is novel.

Recently, work performed in collaboration with Harvard Medical School has shown that the Drosophila expression system is able to produce protein with native-like conformation as determined by X-ray crystallographic studies (Modis et al., PNAS USA (2003) 100:6986-6991; Modis et al, Nature (2004) 427(6972)313-319; and Modis et al, J. Virol. (2005) 79(2):1223-1231). In addition to producing high quality antigens, the inventors have developed methods of purification that allow for the purification of the proteins without damaging the quality of the proteins. The use of high quality Drosophila S2-cell expressed immunogen means: 1) much less protein is needed to produce a robust immune response, 2) the quality of the immune response is increased, and 3) the efficacy of subunit vaccines is improved.

There is a clear need for new technologies that can be used to respond quickly to influenza outbreaks and pandemics, to produce sufficient doses of high quality and safe vaccine for all populations (including the immunodeficient population), and to produce improved vaccine formulations with increased immunogenicity and efficacy. Some of the technical problems to be solved are engineering nucleotide sequences for immunogenic and protective epitopes, expression and purification of the subunit proteins encoded by the nucleotide sequences through methods that can be scaled up to commercial production, and determining which adjuvants, if any, should be included in vaccine formulations containing the subunit proteins. The invention disclosed herein meets the need of developing a new influenza vaccine production method and solves associated technical problems.

### SUMMARY OF THE INVENTION

The invention provides recombinant influenza subunit proteins and immunogenic compositions that can be utilized as vaccines to afford protection against influenza in animal models and humans. The recombinant subunit proteins of the invention are expressed from stably transformed insect cells that contain integrated copies of the appropriate expression cassettes in their genome. The insect cell expression system provides high yields of recombinant subunit proteins with native-like conformation. The recombinant subunit proteins of the invention represent full length or truncated forms of the native influenza proteins. Specifically, the subunits are derived from the HA and M1 proteins of influenza. More specifically the subunit proteins are secreted from the transformed insect cells and then purified from the culture medium following the removal of the host cells. Avoiding lysis of the host cells by either viral means or by physical means simplifies purification, improves yields, and avoids potential degradation of the target protein.

The invention also provides for the use of adjuvants as components in an immunogenic composition compatible with the purified proteins to boost the immune response resulting from vaccination. One or more preferred adjuvants are selected from the group comprising saponins (e.g, GP-0100), or derivatives thereof, emulsions alone or in combination with carbohydrates or saponins, and aluminum-based adjuvants (collectively, “alum” or “alum-based adjuvants”) such as aluminum hydroxide, aluminum phosphate, or a mixture thereof. Aluminum hydroxide (commercially available as “Alhydrogel”) was used as alum in the Examples. A saponin is any plant glycoside with soapy action that can be digested to yield a sugar and a sapogenin aglycone. Sapogenin is the nonsugar portion of a saponin. It is usually obtained by hydrolysis, and it has either a complex terpenoid or a steroid structure that forms a practicable starting point in the synthesis of steroid hormones. The saponins of the invention can be any saponin as described above or saponin-like derivative with hydrophobic regions, especially the strongly polar saponins, primarily the polar triterpensaponins such as the polar acidic bisdesmosides, e.g. saponin extract from Quillsjabark Araloside A, Chikosetsusaponin IV, Calendula-Glycoside C, chikosetsusaponin V, Achyranthes-Saponin B. Calendula-Glycoside A, Araloside B, Araloside C, Putranjia-Saponin III, Bersamasaponiside, Putrajia-Saponin IV, Trichoside A, Trichoside B, Saponaside A, Trichoside C, Gypsoside. Nutanoside, Dianthoside C, Saponaside D, aescine from Aesculus hippocastanum or sapoalbin from Gyposophilla struthium, preferably, saponin extract Quillaja saponaria Molina and Quil A. In addition, saponin may include glycosylated triterpenoid saponins derived from Quillaja Saponaria Molina of Beta Amytin type with 8-11 carbohydrate moieties as described in U.S. Pat. No. 5,679,354. Saponins as defined herein include saponins that may be combined with other materials, such as in an immune stimulating complex (“ISCOM”)-like structure as described in U.S. Pat. No. 5,679,354. Saponins also include saponin-like molecules derived from any of the above structures, such as GPI-0100, such as described in U.S. Pat. No. 6,262,029. Preferably, the saponins of the invention are amphiphilic natural products derived from the bark of the tree, Quillaia saponaria. Preferably, they consist of mixtures of triterpene glycosides with an average molecular weight (M_{W}) of 2000. A particularly preferred embodiment of the invention is a purified fraction of this mixture.

The invention further provides methods for utilizing the vaccines to elicit the production of antibodies against the various types and subtypes of influenza virus in a mammalian host as a means of conferring protection against influenza. The vaccine formulations are shown to induce strong overall antibody titers, as well as strong hemagglutinin-inhibition antibody titers, in comparison to other formulations. Furthermore, the vaccine formulations are shown to provide protection against influenza challenge in a mouse model. In comparison to conventionally produced influenza immunogens, the proteins produced by the invention have increased immunogenicity and efficacy, are less costly to produce, and have a shorter production cycle.

### BRIEF DESCRIPTION OF THE DRAWINGS

FIG. 1. Lymphocyte proliferation of antigen stimulated splenocytes

FIG. 2. IFN-γ production from antigen stimulated splenocytes.

FIG. 3. IL-5 production from antigen stimulated splenocytes.

FIG. 4. H5 HA ELISA antibody titers.

FIG. 5. H3 HA ELISA antibody titers.

### DETAILED DESCRIPTION OF THE INVENTION

The invention provides influenza recombinant subunit proteins that are produced and secreted from stable insect cell lines that have been transformed with the appropriate expression plasmid. The recombinant proteins are used individually or combined together with or without adjuvant(s) such that they are effective in inducing a strong antibody response capable of inhibiting hemagglutination in in vitro assays. This antibody response is indicative of in vivo protection against influenza infection. When used in combinations, in addition to inducing relevant antibody responses, the recombinant proteins also induce cellular immune responses which further enhance the efficacy of the vaccine formulation. The use of appropriate antigens, with or without adjuvants or adjuvant combinations, can be used to induce a specific immune response that results in antibodies that are capable of providing protection from influenza.

In a preferred embodiment of the invention, the recombinant influenza subunit proteins that are a component of the vaccine formulation described herein are produced in a eukaryotic expression system that utilizes insect cells. Insect cells are an alternative eukaryotic expression system that provides the ability to express properly folded and post-translationally modified proteins while providing simple and relatively inexpensive growth conditions. The majority of insect cell expression systems are based on the use of baculovirus-derived vectors to drive expression of recombinant proteins. Expression systems using baculovirus-derived vectors are not based on the use of stable expression cell lines. Instead these systems rely on the infection of host cells for each production cycle. As a result, over-expression of the desired product by the baculovirus vector also results in virus production, which leads to lysis of the host cells. Expression systems based on the generation of stable cell lines via integration of the expression cassettes into the genome of the host cell are capable of being used over multiple generations for the expression of the desired product. This provides a greater level of consistency in the production of a given product. The Drosophila melanogaster expression system (“Drosophila expression system” or “Drosophila system”) (Johansen, H. et al., Genes Dev. (1989) 3:882-889; Ivey-Hoyle, M., Curr. Opin. Biotechnol. (1991) 2:704-707; Culp, J. S., et al., Biotechnology (NY) (1991) 9:173-177) is an insect cell expression system based on the generation of stably transformed cell lines for recombinant protein expression. This insect cell expression system has been shown to successfully produce a number of proteins from different sources. Most importantly, the recombinant proteins produced in this expression system have been shown to maintain structural and functional characteristics of the corresponding native proteins. Examples of proteins that have been successfully expressed in the Drosophila expression system include HIV gp120 (Culp, J. S., et al., Biotechnology (NY) (1991) 9:173-177; Ivey-Hoyle, M., Curr. Opin. Biotechnol. (1991) 2:704-707, human dopamine β-hydrolase (Bin et al, Biochem J. (1996) 313:57-64), human vascular cell adhesion protein (Bernard et al, Cytotechnology (1994) 15:139-144), and dengue envelope glycoprotein (Modis et al., PNAS USA (2003) 100:6986-6991; Modis et al, Nature (2004) 427(6972)313-319; and Modis et al, J. Virol. (2005) 79(2):1223-1231; and Zhang et al, Structure (2005) 12(9):1607-1618). HBI has also determined that subunit proteins produced from the Drosophila expression system produced superior immunogenic material. For example, a comparison of Plaque Reduction Neutralization Titers (PRNT₈₀) between comparable Drosophila-expressed dengue E protein and Pichia-expressed dengue E protein showed ranges of 1:400-1:1600 and <1:10-1:80, respectively for the two systems, using equivalent doses for immunization. In each of these examples, the expression levels of Drosophila expressed proteins were greater than equivalent proteins expressed in other systems that had been utilized and, more importantly, the Drosophila products expressed were of higher quality based on functional and/or structural studies.

In a more preferred embodiment, the insect cells used as host cells for expression of the influenza recombinant subunit proteins are or are derived from the Drosophila melanogaster S2 cell line (Schneider, J Embryol. Exp. Morph. (1972) 27:353-365).

In contrast to other heterologous expression systems that have been used to express subunits for use in influenza vaccine formulations, the Drosophila expression system provides a stable and continuous insect cell culture system that has the potential to produce large quantities of native-like subunit proteins that maintain relevant immunological properties.

While the Drosophila expression system has the potential to produce structurally and immunologically relevant proteins, not all attempts to express heterologous proteins or truncated versions of proteins have met with success. Therefore, a systematic evaluation is required to determine the potential to express a particular heterologous protein subunit in the S2 cell expression system. Examples of proteins and their subunits that have failed to express adequately in the S2 cell system include the dengue and hepatitis C NS3 proteins, truncated forms of the full-length dengue NS1 protein, certain truncated forms of the full length dengue E protein, truncated forms of the full-length malaria LSA-1 protein, and the malaria p19 subunit of the MSP-1 protein.

In addition, specific proteins used for vaccine formulations are subject to the selection of the proper adjuvant and mode of administration for optimal efficacy of the vaccine. For example, alhydrogel will stimulate a good Th2 response in many instances. However, a Th1 response will require that an adjuvant such as GPI-0100 can be used. Combination of these two adjuvants will lead to yet another immune response dependent on the vaccine antigen used. Vaccination via subcutaneous route can work for some vaccines while the intramuscular route can be superior for others.

The focus of the example used to support the present invention is on two specific influenza type A subtypes, H3N2 and H5N1. For work on the H3N2 subtype, the A/Fujian/411/02 influenza strain was used as the source for HA gene sequences. For work on the H5N1 subtype the A/Hong Kong/156/97 was used as the source for HA and M1 sequences. The nucleotide sequences encoding the various proteins of these specific influenza strains as well as most other strains are available in the GenBank (www.ncbi.nlm.nih.gov) and ISD (www.flu.lanl.gov) databases. The same methods used to assemble and express the influenza subunits described above can be extended to all type A influenza subtypes and strains.

In the present invention, the expression and secretion of the influenza subunit proteins HA and M1 from Drosophila S2 cells was evaluated by operably linking the coding sequences of such proteins to a secretion signal sequence such that the expressed products were secreted into the culture medium. For the expression and secretion of HA and M1, the tPA (tissue plasminogen activator) secretion signal was utilized. All nucleotide sequences encoding the described influenza subunit proteins were made synthetically (DNA2.0, Menlo Park, Calif.) and were derived from sequences available in the GenBank and ISD databases. The specific synthetic DNA sequences encoding the influenza subunit proteins were also codon optimized for expression in insect cells. The subunit protein encoding sequences described herein were cloned into Drosophila expression plasmids under the control of the Drosophila MtnA (metallothionein) promoter utilizing standard recombinant DNA methods. The Drosophila expression plasmids containing the cloned influenza sequences were then used to transform Drosophila S2 cells.

In a preferred embodiment, the HA protein was truncated at the C-terminal end to remove the membrane spanning region to allow for secretion of a soluble subunit. The soluble membrane anchor-less subunit is referred to as the HA ectodomain (surface exposed region of a transmembrane anchored protein). The truncated and secreted HA subunits are designed to maintain native-like characteristics of the exposed portion of the membrane anchored HA as displayed on the surface of the virus and are capable of eliciting a strong immune response when combined in a vaccine formulation. The HA ectodomain contains all of the HA1 region and approximately two thirds of the HA2 region (truncation is in the HA2 region). Specifically the H3 HA protein was truncated at amino acid Gly520 and the H5 HA protein was truncated at amino acid Gly521 of the full-length sequences (includes the secretion signal). The C-terminal portion so truncated at amino acid Gly₅₂₀ in the case of H3 HA protein, and at Gly₅₂₁ in the case of H5 HA protein, is called herein a “nominal ectodomain”. The truncation point can be varied up to 10% of the length of a nominal ectodomain so long as such variation does not affect conformation of the epitopes of the remaining soluble HA subunit protein (ectodomain). The native secretion signal sequences were removed for expression as a heterologous secretion signal (tPA) provided by the expression plasmid was utilized to direct secretion of the influenza subunits. The H3 and H5 HA protein sequences expressed are SEQ ID NO:1 and SEQ ID NO:2, respectively. The HA ectodomain subunits are referred to by the HA subtype from which they are derived followed by HA-Ecto, for example H3 HA-Ecto.

In an alternative embodiment, the expression of HA subunits consisting of further truncations of the HA molecule, i.e., truncations that remove a larger amount of the C-terminal end beyond that removed by the ectodomain and segments of the N-terminal end of the HA sequence is described below. These further truncation of HA are designed to express HA subunits that result in a more focused immune response to the naturally exposed surfaces of the HA molecule upon immunization. Such further truncations of the ectodomain are produced by removing the entire HA2 region (the C-terminal region representing approximately one-third of full length HA protein) and a small segments of the N-terminal region of HA. The N- and C-terminally truncated subunits encompass the HA region known as the globular heads and are therefore referred to as HA-heads. The C-terminal truncation is at constant point for all “head” subunits. Specifically the “head” subunits are truncated at Arg₃₂₉ for H3 HA-heads and Arg₃₂₆ for H5 HA-heads (the number of amino acids for this purpose is based on the mature HA protein and does not include the secretion signal). The specified N- and C-terminal truncations for both the H3 and H5 HA-heads are called herein “nominal HA-heads”. Both the N- and C-terminal truncation points can be varied up to 10% of the length of the nominal HA-heads so long as such variation does not affect the conformation of the epitopes on the remaining soluble HA-head. The “head” subunits are distinguished by the position of the N-terminal truncation. For example a subunit named “H3 HA-A19-head” is one derived from the H3 subtype and is N-terminally truncated at Ala₁₉ (A19). Again, the numbering is based on the mature HA protein. The HA-head sequences expressed are shown in Alignments 1 and 2 of Appendix A for H3 and H5 respectively, relative to the corresponding HA ectodomain sequence. Appendix A is fully incorporated herein by reference. The amino acid sequence of H3 HA-A19-head is SEQ ID NO:3. The amino acid sequence of H3 HA-G49-head is SEQ ID NO:4. The amino acid sequence of H5 HA-A9-head is SEQ ID NO:5. The amino acid sequence of H5 HA-G39-head is SEQ ID NO:6.

In a preferred embodiment, the H5N1 M1 subunit representing the full length native M1 protein was expressed. The M1 protein is encoded by amino acids 1 to 252. The M1 protein sequence expressed is shown in SEQ ID NO:7. The amino acid sequences of SEQ ID NOS:1 to 7 can have up to 10% substitution in residues so long as such substitutions do not affect conformation of the epitopes.

The influenza recombinant subunit proteins that are expressed and secreted from the stably transformed S2 cell lines, as described below and utilized in the preferred vaccine formulations, are first purified by a variety of methods, as described below. The preferred purification method produces protein that maintains its native conformation.

In a preferred embodiment, a vaccine formulation that combines the Drosophila-expressed influenza recombinant subunit proteins as described herein, with or without one or more adjuvants, potentiates a strong immune response. The use of such a vaccine formulation induces strong hemagglutinin antibody titers, e.g., ≧1:40. The unique ability of such a vaccine formulation to elicit high hemagglutinin antibody titers is supported by the fact that other recombinantly expressed influenza proteins failed to induce potent immune responses. Furthermore, the vaccine formulation is capable of conferring protection from influenza challenge in the mouse model. Further details that describe the characteristics of the individual components and the remarkable efficacy of this vaccine formulation are contained below.

In another embodiment, the vaccine formulation is characterized by the use of low doses of recombinant subunit proteins capable of eliciting a specific and potent immune response. Low doses are defined as 15 μg or less of recombinant protein. This is in contrast to other influenza recombinant subunit proteins that have required higher doses to achieve moderate immune responses.

The present invention thus concerns and provides a vaccine formulation as a means for preventing or attenuating infection by influenza viruses. As used herein, a vaccine is said to prevent or attenuate disease if its administration to an individual results either in the total or partial immunity of the individual to the disease, i.e., a total or partial suppression of disease symptoms.

To immunize subjects against influenza, a vaccine formulation containing one or more subunits is administered to a subject by means of conventional immunization protocols involving, usually but not restricted to, multiple administrations of the vaccine. The use of the immunogenic compositions of the invention in multiple administrations may result in the increase of antibody levels and in the diversity of the immunoglobulin repertoire expressed by the immunized subject.

Administration of the immunogenic composition is typically by injection, e.g., intramuscular or subcutaneous; however, other systemic modes of administration may also be employed.

According to the present invention, an “effective dose” of the immunogenic composition is one that is sufficient to achieve a desired biological effect. Generally, the dosage needed to provide an effective amount of the composition will vary depending upon such factors as the subject's age, genetic background, condition, and sex. The immunogenic preparations of the invention can be administered by either single or multiple dosages of an effective amount. Effective amounts of the compositions of the invention can vary from 1-100 μg per dose, more preferably from 1-15 μg per dose.

Although the descriptions presented above and the Examples that follow are primarily directed at the expression of the influenza subunits HA and M1 from the type A subtypes H3N2 and H5N1, the methods and vaccine formulation can be applied to other type A subtypes and to influenza types B and C.

### EXAMPLES

The Examples below demonstrate the effective expression of the influenza subunit proteins HA and M1 proteins utilizing stably transformed insect cell lines. For the purpose of these Examples, the Drosophila expression system is utilized. The purification of the expressed recombinant subunit proteins is also demonstrated.

The Examples further demonstrate that the Drosophila expressed recombinant proteins when used as immunogens result in robust and biologically relevant immune responses. The results presented demonstrate that individual influenza subunit proteins derived from the native influenza proteins HA and M1 or various combinations of these same subunit proteins are capable of providing enhanced protection from challenge in mouse models. Thus, the utilization of recombinantly expressed HA and M1 proteins from stably transformed insect cells results in superior immunogenic compositions and meets the need and solves the technical problem set forth above.

### Example 1

### Expression and Purification of Influenza HA Ectodomains from H5N1 and H3N2 Subtypes

A series of expression plasmids designed for the expression and selection of heterologous recombinant target proteins in cultured Drosophila cells was utilized for the work described. For details about the preparation of the expression plasmids, see U.S. Pat. Nos.: 5,550,043; 5,681,713; 5,705,359; and 6,046,025, the contents of which are fully incorporated herein by reference. Specifically, the two plasmids utilized for this work are pMttbns and pCoHygro. The pMttbns expression vector contains the following elements: the Drosophila metallothionein promoter (Mtn), the human tissue plasminogen activator (tPA) signal sequence, and the SV40 early polyadenylation signal (Culp et al, Biotechnology (1991) 9:173-177). The pCoHygro plasmid provides a selectable marker for hygromycin (Van der Straten, Methods in Mol. and Cell Biol. (1989) 1:1-8). The hygromycin gene is under the transcriptional control of the Drosophila COPIA transposable element long terminal repeat. The pMttbns vector was modified by deleting a 15 base pair BamHI fragment which contained an extraneous Xho I site. This modified vector, referred to as pMttΔXho, allows for directional cloning of inserts utilizing unique Bgl II and Xho I sites. For details about the preparation of the expression plasmids and use in the Drosophila expression system, see commonly assigned U.S. Pat. Nos. 6,165,477; 6,416,763; 6,432,411; and 6,749,857, the contents of which are fully incorporated herein by reference. Unless otherwise defined herein, the definitions of terms used in such commonly assigned patents and related to the Drosophila expression system shall apply herein. The DNA sequences cloned into the plasmids in such commonly assigned patents are, of course, different from, and superseded by, the cloned influenza sequences disclosed herein.

The Drosophila expression system has been reported to express high levels of properly folded proteins (Culp et al Biotechnology (1991) 9:173-177, Bernard et al Cytotechnology (1994) 15:139-144, Bin et al Biochem J. (1996) 313:57-64, Incardona and Rosenberry, Mol. Biol. of the Cell (1996) 7:595-611). Expression vectors based on the Drosophila metallothionein (Mtn) promoter provide regulated expression of heterologous proteins (Van der Straten, Methods in Mol. and Cell Biol. (1989) 1:1-8), Johansen, H. et al., Genes Dev. (1989) 3:882-889; and Culp et al Biotechnology (1991) 9:173-177). Selection of stable transformants following co-transformation with a loaded expression plasmid and a plasmid encoding hygromycin resistance (Van der Straten, Methods in Mol. and Cell Biol. (1989) 1:1-8)) results in the stable integration of multiple copies of the target gene carried by (aka “loaded into”) the expression plasmid (Culp et al Biotechnology (1991) 9:173-177). The use of the Drosophila expression system utilizing Mtn expression plasmids allows for the generation of stable transformants that can be effectively maintained and are capable of expressing proteins of high quality and at high yields. Expression is induced by the addition of copper sulfate.

The Drosophila expression plasmids encoding the influenza subunit proteins were constructed by inserting defined segments of the appropriate genes in the Drosophila expression vector pMttΔXho. The appropriate regions of the influenza genes were generated by gene synthesis (DNA2.0, Menlo Park, Calif.). In addition to the synthesis of appropriate genes of interest, the genes were also codon optimized for expression in insect cells. The synthetic genes also included appropriate restriction endonuclease cleavage sites for cloning along with necessary control elements, such as stop codons. The synthetic influenza genes were cloned into the pMttΔXho vector digested with BglII and XhoI. Cloning into the Bgl II site of pMttΔXho results in the addition of four amino acids, Gly-Ala-Arg-Ser, to the amino terminus of the protein expressed due to the fusion with the tPA secretion signal sequence. All of the constructs were sequenced to verify that the various components that have been introduced were correct and that the proper reading frame had been maintained.

Drosophila S2 cells (Schneider, J. Embryol. Exp. Morph. (1972) 27:353-365) obtained from ATCC were utilized in the S2 system. Cells were adapted to growth in Excell 420 medium (JRH Biosciences, Lenexa, Kans.) and all procedures and culturing described herein were in Excell 420 medium. Cells were passed between days 5 and 7 and were typically seeded with expression plasmids at a density of 1×10⁶ cells/ml and incubated at 26° C. Expression plasmids containing sequences encoding influenza subunit proteins were transformed into S2 cells by means of the calcium phosphate method. The cells were co-transformed with the pCoHygro plasmids for selection with hygromycin B at a ratio of 20 μg of expression plasmid to 1 μg of pCoHygro. Following transformation, cells resistant to hygromycin, 0.3 mg/ml, were selected. Once stable cell lines were selected, they were evaluated for expression of the appropriate products. Five ml aliquots of culture medium were seeded at 2×10⁶ selected cells/ml, induced with 0.2 mM CuSO₄, and cultured at 26° C. for 7 days. Cultures were evaluated for expression of subunit proteins in both the cell associated fractions and the culture medium. Proteins were separated by SDS-PAGE and either stained with Coomassie blue or blotted to nitrocellulose. Antibodies specific for a given target protein being expressed were used to probe Western blots. Expression levels of 1 mg/L or greater are readily detected in Drosophila cultures by Coomassie staining of SDS-PAGE gels. To produce larger volumes of product, the transformed Drosophila S2 cells were grown as suspension cultures in spinner flasks or bioreactors.

The full length HA gene (HA0) of the H3N2 strain A/Fujian/411/02 encodes a protein of 566 amino acid residues. Specifically, the sequence utilized was derived from the nucleotide sequence in accession number ISDN38157 (ISD, www.flu.lanl.gov). The non-truncated protein sequence contains a 16 amino acid secretion signal sequence at the N-terminus and a C-terminal membrane anchor. For the expression of a soluble H3 HA ectodomain (H3 HA-Ecto) an N- and C-truncated molecule was expressed that is contained in the sequence from Gln₁₇ to Gly₅₂₆ (residue 175 of HA2, analogous to the C-terminus of the X31 crystal structure, Wilson et al. Nature (1981) 289:366-373) of the full length protein.

The pMttΔXho expression plasmid containing (“loaded with”) the synthetic gene for the H3 HA-Ecto subunit protein was used to transform S2 cells. Upon selection of stable cell lines the cells were screened for expression of the secreted form of the H3 HA-Ecto protein. The expression of the described H3 HA-Ecto subunit resulted in a uniform product of the expected molecular weight. The glycosylation pattern of the secreted H3 HA-Ecto is uniform as the treatment with PNGase results in a shift that is consistent with the presence of 7 glycosylation sites. The expression level of the H3 HA-Ecto target protein secreted into the culture medium of S2 cells has been estimated to be between 30 and 40 μg/ml.

The full length HA gene (HA0) of the A/Hong Kong/156/97 (H5N1) strain encodes a protein of 568 amino acid residues. Specifically, the sequences utilized are derived from the nucleotide sequence in accession number AF046088 (Genbank, www.ncbi.nlm.nih.gov). The HA0 protein sequence contains a 16 amino acid secretion signal sequence at the N-terminus and a C-terminal membrane anchor. For the expression of a soluble H5 HA molecule (ectodomain), an N- and C-truncated molecule was expressed that is contained the sequence from Asp₁₇ to Gly₅₂₁ (residue 175 of HA2, analogous to the C-terminus of the X31 crystal structure, Wilson et al. Nature (1981) 289:366-367), of the full length protein. The HA of the A/Hong Kong/156/97 (H5N1) strain contains a stretch of 6 basic amino acid residues at the HA1/HA2 junction that encodes a furin cleavage site. This site is cleaved upon expression in S2 cells.

The pMttΔXho expression plasmid containing (“loaded with”) the synthetic gene for the H5 HA-Ecto subunit protein was used to transform S2 cells. Upon selection of stable cell lines the cells were screened for expression of the secreted form of the H5 HA-Ecto protein. The expression of the described H5 HA-Ecto subunit resulted in a product consisting of a number of bands (+ or −10 kD) in the range of the expected molecular weight under non-reducing conditions. As the glycosylation pattern of the secreted H5 HA-Ecto appeared to be uniform based on the treatment with PNGase which results in a shift that is consistent with the presence of 5 glycosylation sites under reducing conditions. Therefore, the multiband pattern of expression appears to be the result of variations in folding of the molecule. The expression level of the H5 HA-Ecto target protein secreted into the culture medium of S2 cells has been estimated to be approximately 5 μg/ml.

The HA protein for the H5N1 strain contains a stretch of basic amino acid residues at the HA1/HA2 junction that encodes a furin cleavage site. This site is cleaved upon expression in S2 cells. An alternative form of the H5 HA-Ecto was also expressed. This alternative form was made by creating a mutation within the furin cleavage site which prevented the protease cleavage of the H5 HA-Ecto subunits upon expression. The eight amino acid sequence, Arg₃₃₉-Glu-Arg-Arg-Arg-Lys-Lys-Arg of the Hong Kong strain which contains a furin cleavage site (Arg-Lys-Lys-Arg) was removed and replaced by the four amino acid sequence Lys-Gln-Thr-Arg. The mutated forms of the H5 HA ectodomain is referred to as H5-HK-HA-Ecto-mut.

The pMttΔXho expression plasmid containing the synthetic gene for the H5 HA-Ecto-mut subunit was used to transform S2 cells. Upon selection of stable cell lines the cells were screened for expression of the secreted form of the H5 HA-Ecto-mut protein. The expression of the H5 HA-Ecto-mut subunit resulted in a more uniform protect than that of the H5 HA-Ecto subunits. The expression level of the H5-HK-HA Ecto protein secreted into the culture medium of the S2 cultures has been estimated to be 5 to 10 μg/ml.

Standard chromatographic methods were used to separate the secreted recombinant influenza HA subunit proteins from the S2 culture supernatant. In order to produce materials for human therapy, the methods development effort was influenced by the need to create methods that could be feasibly scaled and used as a current Good Manufacturing Practice (“cGMP”) production process. Based upon the inventors' past success with immunoaffinity chromatography (“IAC”), this method was a primary focus of the inventors' development efforts. As is known in the art, an important criterion for choosing antibodies for use in purification is availability of either the relevant hybridomas, or the antibody itself, being available in bulk, which limited the reagents that could be evaluated for use in IAC.

Non-immunoaffinity purification approaches such as the method of Vanlandschoot et al. (Arch. Virol. (1996) 141:1715-1726), which was originally used to purify A/Victoria/3/75 (H3N2) HA expressed as a secreted product in Spodoptera frugiperda-9 (Sf9) cells, were also evaluated for the purification of secreted influenza HA-Ecto subunit from the S2 culture supernatant. For H3 HA-Ecto subunit a two step purification method was developed. The bulk harvest was diluted ⅓ with buffer A (20 mM sodium phosphate, pH 7.0) then loaded onto a SP-sepharose (GE Healthcare, Piscataway, N.J.) column, which was subsequently washed with wash buffer B (50 mM sodium phosphate, pH 7.0) until baseline absorbance was achieved. Bound H3 HA-Ecto was eluted with buffer B containing 0.5M NaCl. The elution product from the SP-sepharose was then diluted ½ with buffer C (0.1M sodium phosphate, pH 7.0) then loaded onto a ceramic hydroxyapatite column (CHT; Bio-Rad Laboratories, Hercules, Calif.), which was then washed with buffer C until baseline absorbance was achieved. Bound H3 HA ectodomain was eluted with 0.5M sodium phosphate, pH 7.0. The product was concentrated and buffer exchanged by ultrafiltration for characterization.

The H5 HA-Ecto subunits were purified by a three step chromatographic process. The bulk harvest was diluted ¼ with buffer A (25 mM Tris-HCl, pH 8.8, +0.05% tween-20) then loaded onto a CHT column, which was subsequently washed with buffer A until baseline absorbance was achieved. Bound H5 HA-Ecto was eluted with 50 mM sodium phosphate, pH 7.45, +0.05% tween-20. The elution product was loaded onto a Q-sepharose (GE Healthcare, Piscataway, N.J.) column equilibrated against buffer A. The column was washed with buffer A then with buffer A containing 50 mM NaCl. The bound H5 HA-Ecto was eluted with buffer A containing 1M NaCl. The Q-sepharose product was further fractionated by size exclusion chromatography on a Sephacryl S-100 column (1.5×95.5 cm) using 11 mM phosphate buffered saline (140 mM NaCl), pH 7.2, for column buffer. The fractions containing H5 HA-Ecto were pooled and concentrated for characterization.

### Example 2

### Expression and Purification of Influenza HA “Heads” from H3N2 and H5N1 Subtypes

In an effort to express a soluble form of HA capable of eliciting a more focused immune response, the ectodomain subunits described in Example 1 were further truncated at both the N- and C-terminal ends. The N- and C-terminally truncated subunits encompass the HA region known as the globular heads and are therefore referred to as HA-heads. The C-terminal truncation is at constant point for all “head” subunits. Specifically the “head” subunits are truncated at Arg₃₂₉ for H3 HA-heads and Arg₃₂₆ for H5 HA-heads (the number of amino acids for this purpose is based on the mature HA protein—does not include the secretion signal—as opposed to the numbering in Example 1 which is based on the full length sequence containing the secretion signal). Two N-terminal truncations were made for both H3- and H5-heads. While the numbering of the truncations between the two subtypes does not match, the truncations are equivalent based on alignment of the protein sequences. The first N-terminal truncation is made at an Ala residue, Ala₉ for H5 and Ala₁₉ for H3. The second N-terminal truncation is made at a Gly residue, Gly₃₉ for H5 and Gly₄₉ for H3. The “head” subunits are designated by the position of the N-terminal truncation, specifically for the above described truncations the subunits are referred to as H5 HA-A9-head, H5 HA-G39, H3 HA-A19-head, and H3-HA-G49-head.

The methods used to clone, transform, express and characterize the HA-head subunits are the same as those described in Example 1. Upon selection of stable cell lines, the cells were screened for expression of the secreted form of the HA-heads. The expression of the described HA-head subunits resulted in a uniform product of the expected molecular weight for H5 derived heads where as expression of H3 derived heads resulted in multiple bands in the a range (+ or −10 kD) of the expected molecular weight. The expression level of the H3 HA-heads and H5 HA-heads secreted into the culture medium of the S2 cultures has been approximately 5 μg/ml and 20 μg/ml, respectively.

Purification of H5 HA-heads was accomplished by a non-immunoaffinity purification method. Bulk harvest was diluted ⅓ in buffer A (20 mM sodium phosphate, pH 6.2) then loaded onto a CHT column, which was washed with buffer A until baseline absorbance was achieved. The unbound material in the flow-through, which contained the H5 HA-heads, was loaded directly onto a SP-sepharose column, which was washed with buffer A until baseline absorbance was achieved. Bound H5 HA-heads were eluted with buffer A containing 0.1M NaCl. The elution product was then polished by size exclusion chromatography on a Sephacryl S-100 (GE Healthcare, Piscataway, N.J.) column (1.5×95.5 cm) using 11 mM phosphate buffered saline (140 mM NaCl), pH 7.2, for column buffer. The fractions containing H5 HA-heads were pooled and concentrated for characterization.

### Example 3

### Expression and Purification of Influenza M1 from H5N1 Subtype

The full length M1 gene from the H5N1 strain A/Hong Kong/156/97 encodes a protein of 252 amino acids. M1 is derived from the influenza M sequence that also encodes the nucleotide sequence for the M2 protein. The sequence encoding Met₁ to Lys₂₅₂ from the M sequence was used to express M1 protein in S2 cells. This sequence was derived from the nucleotide sequence for the H5N1 M sequence contained in accession number AF046090 (GenBank, www.ncbi.nlm.nih.gov). Although the M1 protein is not one that is normally secreted from the cell, for this work the M1 protein, as defined above, was linked to the tPA secretion signal of the Drosophila expression plasmid to produce a secreted form of the truncated M protein.

The methods used to clone, transform, express and characterize the M1 protein are those described in Example 1. Upon selection of stable cell lines, the cells were screened for expression of the secreted form of the H5N1 M1 target protein. The expression of the described M1 subunit resulted in a uniform product of the expected molecular weight. The expression level of the H5N1 M1 protein secreted into the culture medium of the S2 cultures has been estimated to be 15 to 20 μg/ml.

Unlike HA, chromatographic purification methods for M1 protein have not been reported in the literature, with the exception of nickel chelation columns for purification of His-tagged recombinant M1 proteins (Hara et al., Microbiol. Immunol. (2003) 47:521-526; Watanabe et al., J. Virol. (1996) 70:241-247). To maintain native conformation of M1, the addition of a His-tag is not preferred. Other methods for purification of M1 have been acid-chloroform-methanol extraction (Gregoriades, Virology (1973) 54:369-383) and acid-dependent detergent extraction (Zhirnov, Virology (1992) 186:327-330), neither of which is well suited for production purposes. As for the HA protein, IAC using monoclonal antibodies is a preferred method of purifying M1 protein.

Alternative methods of purification were also evaluated leading to the development of a non-immunoaffinity purification method. The bulk harvest was diluted ½ with 2M sodium sulfate then loaded onto a phenyl sepharose (GE Healthcare, Piscataway, N.J.) column equilibrated with 1M sodium sulfate. The column was washed with 1M sodium sulfate until baseline absorbance was achieved then bound material was eluted with deionized water. The water eluent was loaded directly onto a SP-sepharose column equilibrated in buffer A (10 mM sodium phosphate, pH 5.5) containing 150 mM NaCl. The column was washed with buffer A containing 150 mM NaCl until baseline absorbance was achieved. Bound material was eluted by a step gradient comprised of buffer A containing 0.5M and 1M NaCl. M1 protein was eluted in the 0.5M NaCl step and was subsequently further purified by size exclusion chromatography on a Sephacryl S-100 column (1.5×94 cm) using 11 mM phosphate buffered saline (140 mM NaCl), pH 7.2, as column buffer. The fractions containing M1 protein were pooled and concentrated by ultrafiltration for characterization.

### Example 4

### Mouse Immunogenicity Study #1 Immunogenicity of S2 expressed H5 HA-heads with and without H5N1 M1 in Balb/c mice

The immunogenicity of H5 antigens expressed and purified according to the invention was evaluated in Balb/c mice. H5 HA-A9-heads with or without H5N1 M1 protein were tested for immunogenic potential. Groups of 5-9 female Balb/c mice aged 6-8 weeks were immunized by the subcutaneous route with the recombinant antigen(s) or appropriate controls as detailed in Table 1 below. Vaccines were delivered as a formulation of antigen(s) with GPI-0100 (250 μg/dose) as adjuvant in a total volume of 0.2 ml. Animals received 2 doses of vaccine at a 4 week interval. Seven days after the last dose of vaccine 4 mice/group were euthanized and spleens collected for analysis of cellular immune responses as described below. Two weeks after the last dose of vaccine, the remaining animals were euthanized and serum samples collected. Humoral responses were assessed based on individual titers of antibodies specific to the immunogen(s), as determined by ELISA antigen binding. In addition, pools of sera were prepared using equivalent volumes of serum from each animal within a group and tested for hemagglutination inhibition (HI) titers.

TABLE 1
Mouse Immunogenicity Study Design using H5 HA-heads Expressed in
Drosophila S2 Expression System

| | Adjuvant | | Dose | |
|---|---|---|---|---|
| Group | (250 μg) | Vaccine Antigen(s) | of Antigen (μg) | # mice |
| 1 | GPI-0100 | H5 HA heads | 3 | 5 |
| 2 | GPI-0100 | H5 HA heads + H5 M1 | 3 | 9 |
| | | | (each antigen) | |
| 3 | GPI-0100 | None | 0 | 9 |

ELISA assays: Antibodies to the influenza proteins (H5 HA heads and H5 M1 proteins) were titrated by an ELISA technique, using a microplate format with wells coated with the specific antigen. Following coating, the wells were blocked with a serum or albumin containing buffer, and then standard ELISA steps were conducted with an alkaline-phosphatase or peroxidase conjugated secondary antibody.

HI assays: HI assays were performed as described by standard methods (Kendal et al., CDC (1982) pB-17-B35) at Southern Research Institute (Frederick, Md.).

Complement fixation assays: Mouse sera were tested for complement fixation activity with the influenza antigens using a quantitative microcomplement fixation assay. Briefly, commercially obtained complement (guinea pig serum), hemolysin (rabbit anti-sheep erythrocyte stromata serum), and sheep erythrocytes (Cedarlane Laboratories, Hornby, Ontario, Canada) were used as the test indicator system and optimal concentrations for use determined by preliminary titrations (Lieberman, et al., Infect. Immunol. (1979) 23:509-521). Dilutions of the purified antigens and mouse antisera were mixed and incubated with diluted complement in buffer on ice for 16 hrs. Controls in which antigen or antiserum were omitted were included. Sheep erythrocytes sensitized by prior incubation with hemolysin were then added to the antigen+antiserum+complement mixture and incubated at 37° C. for 60 min. The reaction mixtures were centrifuged and the absorbance of the supernatants at 413 nm determined. The extent of hemolysis obtained is inversely proportional to the degree of complement fixation by the antigen/antiserum combination, and the dilution of antiserum yielding 50% complement fixation can be determined. Thus, the complement fixing activities of different antisera to influenza antigens were directly compared.

Splenocyte preparations: Splenectomies were performed 7 days post dose 2 on 4 mice each from groups 2 and 3. Splenocyte suspensions were prepared from each mouse spleen, erythrocytes lysed with NH₄Cl, and the final cell pellet washed and resuspended in cell culture medium. Cell counts were performed on each suspension using a Coulter counter, and suspensions diluted to 2×10⁶ cells/ml with culture medium. Splenocytes from individual mice were cultured separately.

Lymphocyte proliferation assays: Aliquots (0.1 ml) of each splenocyte suspension were dispensed into wells of a 96-well cell culture plate. The respective antigens were then added to the wells containing each of the cell suspensions (in quadruplicate) at a final concentration of 5 μg/ml (final volume of 0.2 ml/well). Wells with unstimulated (antigen omitted) cell suspensions were also included. Cultures were incubated at 37° C./5% CO₂/humidified for 7 days, and then one microcurie of tritiated (methyl-³H) thymidine (6.7 Ci/mmol; ICN Biomedicals, Inc., Irvine, Calif.) was added to each well (in a volume of 0.01 ml), and incubation continued for 18 hrs. After that period of time, the cell cultures were harvested onto a glass fiber filtration plate and washed extensively using a vacuum driven harvester system (Filtermate, Perkin Elmer Life Sciences Co., Boston Mass.). The filtration plate was then analyzed for radioactivity in the TopCount Microplate Scintillation and Luminescence Counter (Perkin Elmer Life Sciences Co., Boston Mass.).

Cytokine production assays: Aliquots (0.5 ml) of each splenocyte suspension were dispensed into wells of a 24-well cell culture plate. Five μg of the same antigens used for lymphocyte proliferation were then dispensed into the wells containing each of the cell suspensions (final volume of 1.0 ml/well). Unstimulated cell suspensions were tested as well as controls. Cultures were incubated for 4 days at 37° C./5% CO₂/humidified. The culture supernatants were then harvested and frozen for analysis for specific cytokines. Cytokines in splenocyte culture supernatants were assayed using a flow cytometric bead array assay (BD Biosciences Pharmingen Corp., San Diego Calif.).

TABLE 2
HI Antibody Titers Induced by H5 HA-heads in Balb/c Mice

| | | | Antigen Dose | |
|---|---|---|---|---|
| Group | Adjuvant | Vaccine Antigen | (μg) | HI titer |
| 1 | GPI-0100 | H5 HA-heads | 3 | 20 |
| 2 | GPI-0100 | H5 HA-heads + | 3 | 254^{a} |
| | | H5N1 M1 | (each antigen) | |
| 3 | GPI-0100 | None | 0 | <10 |

^{a}GMT of triplicate assays with individual titers of 320, 320, and 160.

TABLE 3
ELISA Antibody Titer Induced by H5 HA-heads in Balb/c Mice

| Group # | Mouse # | HA-heads titer | M1 titer |
|---|---|---|---|
| 1 | 1 | 30148 | <50 |
| | 2 | 6335 | <50 |
| | 3 | 43365 | <50 |
| | 4 | 15091 | <50 |
| | 5 | 11231 | <50 |
| 2 | 1 | 8054 | 1774 |
| | 2 | 20604 | 3881 |
| | 3 | 20033 | 5315 |
| | 4 | 16708 | 3558 |
| | 5 | 18462 | 4321 |
| 3 | all | <50 | <50 |

The results of the antibody titrations show that good ELISA antibody titers were induced by all antigens. HI antibody titers were raised when mice were immunized with HA protein and particularly high titers (>10 fold higher) were induced when mice were immunized with both HA and M1 proteins.

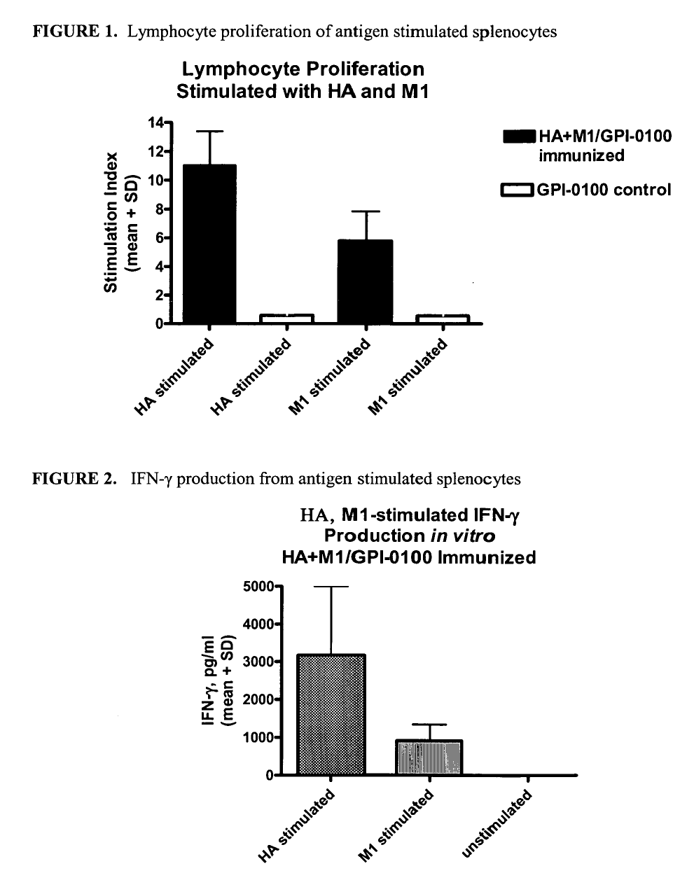

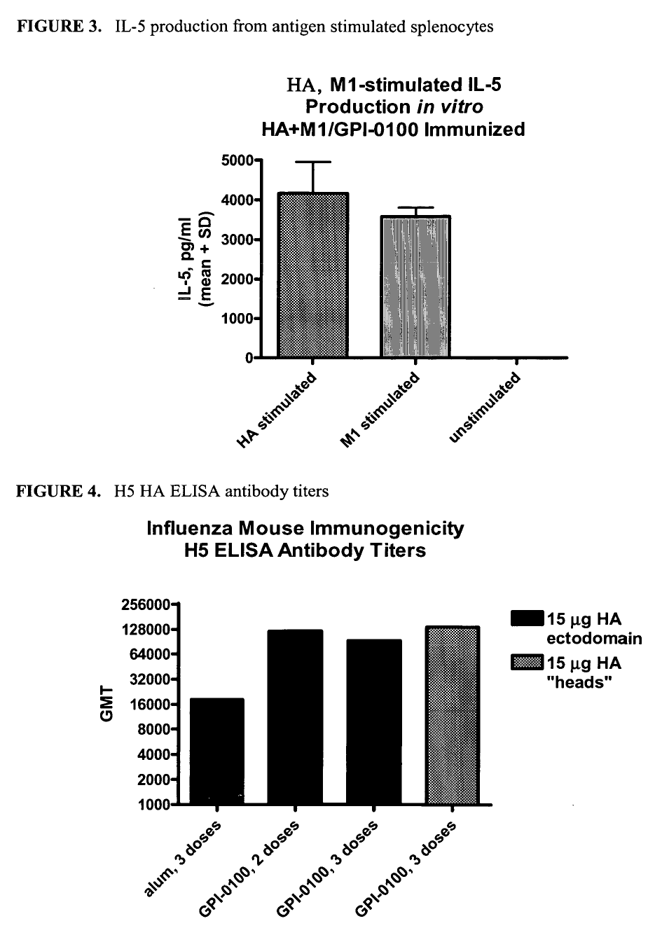

The results of the lymphocyte proliferation (FIG. 1) and the cytokine production assays (FIGS. 2 and 3) demonstrate that the influenza antigens are capable of eliciting good cellular immune responses. When immunized with both antigens and GPI-0100 adjuvant, mice were capable of responding to stimulation with either antigen in vitro by proliferation and production of IFN-γ and IL-5 (as well as TNF-α, IL-2, and IL-4; data not shown). This cell-mediated immune response may be important in providing protective immunity against influenza to specific populations of subjects, such as elderly individuals (McElhaney J E, et al., J. Immunol. 176:6333-6339, 2006).

### Example 5

### Mouse Immunogenicity Study #2 Immunogenicity of S2 expressed H5 HA-Ecto and H5 HA-head subunits in Balb/c mice

The immunogenicity of S2 expressed H5 HA subunit proteins, specifically H5 HA-Ecto-mut and H5 HA-A9-head, were evaluated in Balb/c mice. Groups of 5-10 female Balb/c mice aged 6-8 weeks are immunized by the intramuscular route with the recombinant antigens or appropriate controls. Vaccines were delivered as a formulation of antigen(s) with or without alhydrogel (0.5 mg/dose) or GPI-0100 (250 μg/dose) as adjuvant in a total volume of 0.2 ml. Animals received 2 doses of vaccine at a 4 week interval or 3 doses of vaccine at a 4 week interval between the first 2 doses and a 6 week interval between the 2^{nd} and 3^{rd} doses as indicated in Table 4 below. Two weeks after the last dose of vaccine, animals were euthanized and serum samples tested for reactivity with recombinant proteins by ELISA as described previously in Example 4. Results are shown in FIG. 4.

TABLE 4
Design of Immunogenicity Study Evaluating H5 HA Molecules in Balb/c
Mice

| Group | Adjuvant | Vaccine Antigen and Dose (μg) | # mice |
|---|---|---|---|
| 1 | Alhydrogel | None | 5^{#} |
| 2 | Alhydrogel | 15 μg H5 ectodomain S2 | 5^{#} |
| 3 | GPI-0100 | None | 5^{#} |
| 4 | GPI-0100 | 15 μg H5 ectodomain S2 | 10* |
| 5 | GPI-0100 | 15 μg H5 HA heads S2 | 5^{#} |

*Five mice per group received 2 immunizations, the other five received 3 immunizations.
^{#}Five mice per group received 3 immunizations

The results of the ELISA antibody titrations with either HA ectodomain or HA “heads” demonstrate that that the recombinant proteins are immunogenic. Particularly high antibody titers can be achieved with either antigen when administered with an adjuvant, and particularly when this adjuvant is GPI-0100. No detectable antibody titers were raised in the adjuvant control groups (data not shown).

### Example 6

### Mouse Immunogenicity Study #3 Immunogenicity of S2 expressed H3 HA-Ecto with and without H5N1 M1 in Balb/c mice

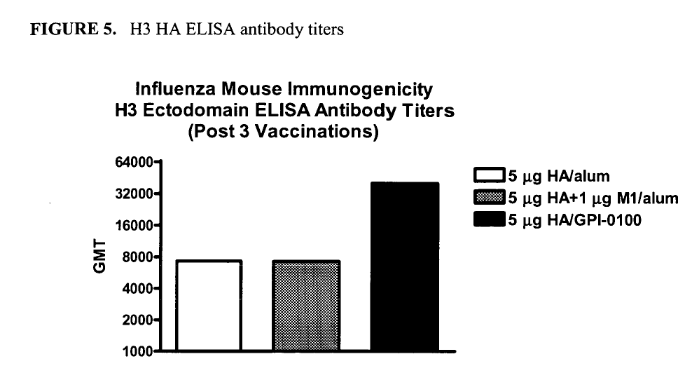

The immunogenicity of S2 expressed H3 HA-Ecto subunits with or without H5 M1 subunits was evaluated in Balb/c mice. Groups of 5-10 female Balb/c mice aged 6-8 weeks were immunized by the intramuscular route with the recombinant antigens or appropriate controls. Vaccines were delivered as a formulation of antigen(s) with or without alum (0.5 mg/dose) or GPI-0100 (250 μg/dose) as adjuvant in a total volume of 0.2 ml. Animals received 2 doses of vaccine at a 4 week interval or 3 doses of vaccine at a 3 week interval as indicated in Table 5 below. Two weeks after the last dose of vaccine, animals were euthanized and individual serum samples tested for reactivity with recombinant proteins by ELISA as described previously in Example 4. Results are shown in FIG. 5.

TABLE 5
Design of Immunogenicity Study
Evaluating H3 HA Molecules in Balb/c Mice

| Group | Adjuvant | Vaccine Antigen and Dose (μg) | mice |
|---|---|---|---|
| 6 | Alhydrogel | None | 5 |
| 7 | Alhydrogel | 5 μg H3 HA ectodomain | 5 |
| 8 | Alhydrogel | 5 μg H3 HA ectodomain + 1 μg H5 M1 | 5 |
| 9 | GPI-0100 | None | 5 |
| 10 | GPI-0100 | 5 μg H3 HA ectodomain | 5 |

The results demonstrate that the H3 HA antigen is immunogenic. The immunogenicity is increased when adjuvanted with alum or GPI-0100. The addition of M1 to the immunizing vaccine did not significantly affect the titers to the HA antigen. No detectable antibody titers were raised in the adjuvant control groups (data not shown).

### REFERENCES

Ada G L, and Jones P D. The immune response to influenza infection. Curr. Topics Microbiol. Immunol. 1986; 128:1-54.
Aymard-Henry M, Coleman M T, Dowdle W R, Layer W G, Schild G C, Webster R G. Influenza virus neuraminidase and neuraminidase inhibition test procedures. Bull. WHO 1973; 48:199-202.
Beran J, Prymula R, Chlibek R, Rychly R, Splino M, Douda P, Gal P. Evaluation of reactogenicity and immunogenicity of two influenza vaccines (Vaxigrip and Fluarix) in the season 1996-1997. Centr. Eur. J. Pub. Health 1998; 4:269-273.
Bernard, A. R., T. A. Kost, L. Overton, C. Cavegn, J. Young, M. Bertrand, Z. Yahia-cherif, C. Chabert and A. Mills. Recombinant protein expression in Drosophila cell line: comparison with the baculovirus system. Cytotechnology 1994; 15:139-144.
Beyer W E, Palache A M, de Jong J C, Osterhaus A D. Cold-adapted live influenza vaccine versus inactivated vaccine: systemic vaccine reactions, local and systemic antibody response and vaccine efficacy. A meta-analysis. Vaccine 2002; 20:1340-1353.
Bin L., S. Tsing, A. H. Kosaka, B. Nguyen, E. G. Osen, C. Bach, H. Chan and J. Barnett. Expression of human dopamine β-hydroxylase in Drosophila Schneider 2 cells. Biochem. J. 1996; 313:57-64.
Bridges C B, Thompson W W, Meltzer M I, Reeve G R, Talamonti W J, Cox N J, Lilac H A, Hall H, Klimov A, Fukuda K. Effectiveness and cost-benefit of influenza vaccination of healthy working adults: a randomized controlled trial. JAMA 2000; 284:1655-1663.
Brydak L B. Influenza and Its Prophylaxis, 1998; 1^{st} ed. Springer P W N, Warsaw.
Centers for Disease Control and Prevention. Prevention and control of influenza: recommendations of the Advisory Committee on Immunization Practices (ACIP). Morbid Mortal Weekly Rep 2001; 50(RR-04):1-63.
Cox R J, Brokstad K A and Ogra P. Influenza Virus: Immunity and Vaccination Strategies. Comparison of the Immune Response to Inactivated and Live, Attenuated Influenza Vaccines. Scandinavian Journal of Immunology 2003; 59:1-15.
Culp J. S., Johansen H., Hellmig B., et al. Regulated expression allows high level production and secretion of HIV gp120 envelope glycoprotein in Drosophila Schneider cells. Biotechnology, 1991; 9:173-177.
Danieli T, Pelletier S L, henis Y I, White J M. Membrane fusion mediated by the influenza virus hemagglutinin requires the concerted action of at least three hemagglutinin trimers. J. Cell Biol. 1996; 133:559-569.
Dolin R, Reichman R C, Madore H P, Maynard R., Linton P. N., Webber-Jones J. A controlled trial of amantadine and rimantadine in the prophylaxis of influenza A infection. N. Engl. J. Med 1982; 307:580-584.
Englund J A, Champlin R E, Wyde P R, Kantarjian H., Atmar R. I., Tarrand J., Yousuf H., Regnery H., Klimov A. I., Cox N. J., Whimbey E. Common emergence of amantadine—and ramantadine—resistant influenza A viruses in symptomatic immunocompromised adults. Clin. Infec. Dis. 1998; 26:1418-1424.
Farrell P. J., Lu M., Prevost J., Brown C., Behie L., Iatrou K. High-Level Expression of Secreted Glycoproteins in Transformed Lepidopteran Insect Cells Using a Novel Expression Vector. Biotechnology and Bioengineering. 1998; 60(6): 656-663.
Gregoriades, A. The membrane protein of influenza virion extracted from virus and infected cells with acidic chloroform-methanol. Virology. 1973; 54:369-383
Ghendon Y. Influenza—its impact and control. World Health Stat Q 1992; 45:306.
Halperin S A, Smith B, Mabrouk T, Germain M, Trepanier P, Hassell T, Treanor J, Gauthier R, Mills E L. 2002. Safety and immunogenicity of a trivalent, inactivated mammalian cell culture-derived influenza vaccine in healthy adults, seniors and children. Vaccine 20(7-8):1240-1247.
Hara, K., M. Shiota, H. Kido, K. Watanabe, K. Nagata, and T. Toyoda. 2003. Inhibition of the protease activity of influenza virus RNA polymerase PA subunit by viral matrix protein. Microbiol. Immunol. 47:521-526.
Harper S, Klimov A, Uyeki T, Fukuda K. Influenza. Clin Lab Med. 2002; 22:863-882.
Heinen P P, de Boer-Luijte E A, Bianchi A T. 2002. Respiratory and systemic humoral and cellular immune responses of pigs to a heterosubtype influenza A virus infection. J. Gen Virol. 82(Pt 11):2697-2707.
Hilleman M R. Realities and enigmas of human viral influenza: pathogenesis, epidemiology and control. Vaccine 2002; 20:3068-3087.
Incardona, J. P. and T. L. Rosenberry. Construction and characterization of secreted and chimeric transmembrane forms of Drosophila acetylcholinesterase: a large truncation of the C-terminal signal peptide does not eliminate glycoinositol phospholipid anchoring. Mol. Biol. of the Cell 1996; 7:595-611.
Ivey-Hoyle, M. Recombinant gene expression in cultured Drosophila melanogaster cells. Curr. Opin. Biotechnol. 1991; 2:704-707.
James J M, Zeiger R S, Lester M R, Fasano M. B., Gern J. E., Mansfield L. E., Schwartz H. J., Sampson H. A., Windom H. H., Machtinger S. B., Lensing S. Safe administration of influenza vaccine to patients with egg allergy. J. Pediatr. 1998; 133:624-628.
Johansen H. A., van der Straten R., Sweet R., Otto E., Maroni G., Rosenberg M. regulated expression at high copy number allows production of a growth-inhibitory oncogene product in Drosophila Schneider cells. Genes and Development. 1989; 3:882-889.
Karzon D T. Cytotoxic T cells in influenza immunity. Semin. Virol. 1996; 7:265-271
Katz, J M, Plowden J, Renshaw-Hoelscher M, Lu X, Tumpey T M, Sambhara S. Immunity of influenza: the challenges of protecting an aging population. Immunol. Res. 2004; 29:113-24.
Kemble G and Greenberg H. Novel generations of influenza vaccines. Vaccine 2003; 21:1789-1795.
Kendal A. P., Shehel J. J., Pereira M. S. Concepts and procedures for laboratory-based influenza surveillance. 1982; p. B17-B35, Centers for Disease Control, Atlanta, Ga.
Kennedy M M. Influenza viral infections: presentation, prevention and treatment. Nurse Pract. 1998; 23:17-28.
Kuroda K, Hauser C, Rott R, Klenk H D, Doerfler W. Expression of influenza virus haemagglutinin in insect cells by a baculovirus vector. EMBO J. 1986; 6:1359-1365.
Lasky T, Terracciano G J, Magder L, Koski C. L., Ballesteros M., Nash D., Clark S., Haber P., Stolley P. D., Schonberger L. B., Chen R. T. The Guillain-Barré syndrome and the 1993 and 1993-1994 influenza vaccines. N. Engl. J. Med. 1998; 339:1797-1802.
Latham and Galarza. Formulation of wild-type and chimeric influenza virus-like particles following simultaneous expression of only four structural proteins. J. Virol. 2001; 75(13):6154-6165.
Lieberman M. M., McKissock D. C., Wright G. L. Passive Immunization against Pseudomonas with a ribosomal vaccine-induced immune serum and immunoglobulin fractions. Infect. Immun., 1979; 23:509-521.
Margolis K L, Nichol K L, Poland G A, Pluhar R. E. Frequency of adverse reactions to influenza vaccine in the elderly: a randomized, placebo controlled trial. JAMA 1990; 264:1139-1141.
McElhaney J E. Overcoming the challenges of immunosenescence in the prevention of acute respiratory illness in older people. Conn. Med. 2003 67:469-74
McElhaney J E, Xie D, Hager W D, Barry M B, Wang Y, Kleppinger A, Ewen C, Kane K P, Bleackley R C. T cell responses are better correlates of Vaccine Protection in the elderly. J. Immunol. 2006; 176:6333-6339.
McMichael A J, Gotch F M, Noble G R, Beare P A S. Cytotoxic T-Cell Immunity to Influenza. N Engl J Med 1983; 309:13-17.
Mishto M, Santoro A., Bellavista E, Bonate M, Monti D, Franceschi C. Immunoproteasomes and immunosenescence. Ageing Res. Rev. 2003 2:419-32
Modis Y, Ogata S, Clements D, Harrison S C. A ligand-binding pocket in the dengue virus envelope glycoprotein. Proc. Natl. Acad. Sci. USA. 2003; 100:6986-6991.
Modis Y, Ogata S, Clements D, Harrison S C. Structure of the dengue virus envelope protein after membrane fusion. Nature. 2004; 427(6972):313-319.
Modis Y, Ogata S, Clements D, Harrison S C. Variable surface epitopes in the crystal structure of dengue virus 3 envelope glycoprotein. J. Virol. 2005; 79(2):1223-1231.
Montgomery D L, Shiver J W, Leander K R, Perry H. C., Friedman A., Martinez D., Ulmer J. B., Donnelly J. J., Liu M. A. Heterologous and homologous protection against influenza A by DNA vaccination: optimization of DNA vectors. DNA Cell Biol. 1993; 12:777-783
Neuzil K M, Reed G W, Mitchel E F, Griffin M. R. Influenza-associated morbidity and mortality in young and middle-aged women. JAMA 1999; 281:901-907.
Nichol K L, Margolis K L, Lind A, Murdoch M., McFadden R., Hauge M., Magnan S., Drake M. Side effects associated with influenza vaccination in healthy working adults: a randomized placebo controlled trial. Arch. Intern. Med. 1996; 156:1546-1550.
Nicholson K G, Wood J M, Zambon M. Influenza. Lancet 2003; 362:1733-1745.
Nicolson C, Major D, Wood J M, Robertson J S. Generation of influenza vaccine on Vero cells by reverse genetics: and H5N1 candidate vaccine strain produced under a quality system. Vaccine 2005; 22:2943-2952.
Palache A M, Scheepers H S, deRegt V, van Ewijk P, Baljet M, Brands R, van Scharrenburg G J. Safety, reactogenicity and immunogenicity of Madin Darby Canine Kidney cell-derived inactivated influenza subunit vaccine. A meta-analysis of clinical studies. Dev. Biol. Stand. 1999; 98:115-125.
Pawelec G. Immunosenescence and human longevity. Biogerontology 2003; 4:167-70
Pawelec G, Barnett Y, Fossey R, Frasca D, Globerson et al., (2002) Front. Biosci. 7:d1056-183.
Pushko P, Tumpey T M, Bu F, Knell J, Robinson R, Smith G. Influenza virus-like particles comprised of the HA, NA and M1 proteins of H9N2 influenza virus induce protective immune responses in BALB/c mice. Vaccine 2005; 23(50):5751-5759.
Saelens X, Vanlandschoot P, Martinet W, Maras M, Neirynck S, Contreras R, Fiers W, Jou W M. Protection of mice against a lethal virus challenge after immunization with yeast-derived secreted influenza virus hemagglutinin. Eur. J. Biochem. 1999; 260(1):166-175.
Schneider I. J. Cell lines derived from late embryonic stages of Drosophila melanogaster. J. Embryol. Exp. Morph., 1972; 27:353-365.
Schoenbaum S C. Economic impact of influenza. Am. J. Med. 1987; 82(Suppl 6A):26-30.
Simonsen L, Clarke M J, Williamson G D, Stroup D. F., Arden N. H., Schonberger L. B. The impact of influenza epidemic on mortality: introducing a severity index. Am. J. Public Health 1997; 87:1944-1950.
Simonsen L, Conn L A, Pinner R W, Teutsch S. Trends in infectious disease hospitalizations in the United States 1980-1994. Arch. Intern. Med. 1998; 158:1923-1928.
Steinhauer D A. Role of hemagglutinin cleavage for the pathogenicity of influenza virus. Virology. 1999; 258:1-20.
Taubenberger J K. Fixed and Frozen Flu: the 1918 Influenza and Lessons for the Future. Avian Diseases 2003; 47(Suppl 3):789-791.
Treanor J J, Wilkinson B E, Masseoud F, Hu-Primmer J, Battaglia R, O'Brien D, Wolff M, Rabinovich G, Blackwelder W, Katz J M. Safety and immunogenicity of a recombinant hemagglutinin vaccine for H5 influenza in humans. Vaccine. 2001; 19:1732-1737.
Ulmer J B, Donnelly J J, Parker S E, Rhodes G. H., Felgner P. L., Dwarki V. J., Gromkowski S. H., Deck R. R., DeWitt C. M., Friedman A., et al. Heterologous protection against influenza by injection of DNA encoding a viral protein. Science 1993; 259:1745-1749.
Van der Straten, A. H., Johansen H., Rosenberg M., Sweet R. W. Introduction and constitutive expression of gene products in cultures of Drosophila cells using hygromycin B selection. Methods in Mol. And Cell. Biol. 1989; 1:1-8.
Vanlandschoot, P., E. Beirnaert, S. Neirynck, X. Saelens, W. Min Jou, and W. Fiers. 1996. Molecular and immunological characterization of soluble aggregated A/Victoria/3/75 (H3N2) influenza haemagglutinin expressed in insect cells. Arch Virol. 141:1715-1726.
Virelizier J L. Host defenses against influenza: The role of anti-hemagglutinin antibody. J. Immunol. 1975; 115:434-439.
Watanabe K, Handa H, Mizumoto K, Nagata, K Mechanism for inhibition of influenza virus RNA polymerase activity by matrix protein. J. Virol. 1996; 70:241-247
Williams J R, Chen P-Y, Cho C T, Chin T D Y. Influenza: Prospect for Prevention and Control. Kaohsiung J. Med. Sci. 2002; 18:421-434.
Wilson I A, Skehel J J, Wiley D C. Structure of the haemagglutinin membrane glycoprotein of influenza virus at 3A resolution. Nature 1981; 289:366-673.
Wood J. M. Selection of influenza vaccine strains and developing pandemic vaccines. Vaccine 2002; 20:B40-B44.
Zhang Y, Zhang W, Ogata S, Clements D, Strauss J H, Baker T S, Kuhn R J, Rossman M G. Conformational changes of the flavivirus E. glycoprotein. Structure. 2004; 12(9):1607-1618.
Zhirnov O P Isolation of matrix protein M1 from influenza viruses by acid-dependent extraction with nonionic detergent. Virology 1992; 186:327-330.

We claim:

1. A method for producing a recombinant subunit influenza vaccine comprising:
expressing and secreting a recombinant influenza hemagglutinin ectodomain protein subunit, wherein the protein subunit lacks a C-terminal transmembrane anchor and is secreted as a soluble protein from stably transformed insect cells; and
formulating said recombinant protein subunit to produce an immunogenic composition that induces the production of hemagglutinin antibody titers in a host vaccinated with the immunogenic composition.

2. A method for producing a recombinant subunit influenza vaccine comprising:
expressing and secreting a recombinant influenza hemagglutinin head protein subunit, wherein the protein subunit lacks a C-terminal transmembrane anchor, lacks an N-terminal portion, and is secreted as a soluble protein from stably transformed insect cells; and
formulating said recombinant protein subunit to produce an immunogenic composition that induces the production of hemagglutinin antibody titers in a host vaccinated with the immunogenic composition.

3. A method for producing a recombinant subunit influenza vaccine comprising:
expressing and secreting a recombinant influenza hemagglutinin ectodomain protein subunit, wherein the protein subunit lacks a C-terminal transmembrane anchor and is secreted as a soluble protein from stably transformed insect cells;
expressing and secreting a recombinant influenza matrix 1 protein subunit, wherein the protein subunit lacks a C-terminal transmembrane anchor, and is secreted as a soluble tetrameric protein from stably transformed insect cells; and
formulating said recombinant hemagglutinin ectodomain and matrix 1 protein subunits to produce an immunogenic composition that induces the production of hemagglutinin antibody titers in a host vaccinated with the immunogenic composition.

4. A method for producing a recombinant subunit influenza vaccine comprising:
expressing and secreting a recombinant influenza hemagglutinin head protein subunit, wherein the protein subunit lacks a C-terminal transmembrane anchor, lacks an N-terminal portion, and is secreted as a soluble protein from stably transformed insect cells;
expressing and secreting a recombinant influenza matrix 1 protein subunit, wherein the protein subunit lacks a C-terminal transmembrane anchor, and is secreted as a soluble tetrameric protein from stably transformed insect cells; and
formulating said recombinant hemagglutinin head and matrix 1 protein subunits to produce an immunogenic composition that induces the production of hemagglutinin antibody titers in a host vaccinated with the immunogenic composition.

5. The method of claim 1, 2, 3, or 4, wherein the influenza virus is influenza A virus.

6. The method of claim 1, 2, 3, or 4, wherein the strain of influenza virus is selected from the group consisting of H5 and H3.

7. The method of claim 1, 2, 3, or 4, wherein the carboxy-terminal portion of the hemagglutinin protein subunit is truncated within 10% of the length of a nominal ectodomain.

8. The method of claim 1, 2, 3, or 4, wherein the stably transformed insect cells are Drosophila melanogaster S2 cells.

9. The method of claim 1, 2, 3, or 4, wherein formulating the immunogenic composition further comprises including in the immunogenic composition one or more adjuvants.

10. The method of claim 1, 2, 3, or 4, wherein formulating the immunogenic composition further comprises including in the immunogenic composition one or more adjuvants selected from the group consisting of saponin and alum.

11. The method of claim 1, 2, 3, or 4, wherein formulating the immunogenic composition further comprises including in the immunogenic composition GPI-0100 adjuvant.

12. The method of claim 1, 2, 3, or 4, wherein formulating the immunogenic composition further comprises including a pharmaceutically acceptable excipient in the immunogenic composition.

13. The method of claim 1, 2, 3, or 4, wherein the protein subunits are purified by immuno-affinity chromatography.

14. The method of claim 1 or 3, wherein the recombinant influenza hemagglutinin ectodomain protein subunit has an amino acid sequence selected from the group consisting of SEQ ID NO:1 and SEQ ID NO:2.

15. The method of claim 2 or 4, wherein the recombinant influenza hemagglutinin head protein subunit has an amino acid sequence selected from the group consisting of SEQ ID NO:3, SEQ ID NO:4, SEQ ID NO:5, and SEQ ID NO:6.

16. The method of claim 2 or 4, wherein the truncation points of the hemagglutinin head protein subunit are selected from the group consisting of N-terminal, C-terminal, and N-terminal and C-terminal, wherein the one or both terminal points can be varied up to 10% of the length of a nominal HA-head.

17. The method of claim 3 or 4, wherein the recombinant influenza matrix 1 protein subunit has an amino acid sequence of SEQ ID NO:7.

18. A method for raising an immunogenic response from a subject, comprising administering in a therapeutically acceptable manner a therapeutically effective amount of the immunogenic composition of claim 1, 2, 3, or 4 to said subject.

19. An immunogenic composition comprising a recombinant subunit influenza vaccine comprising a recombinant influenza hemagglutinin ectodomain protein subunit, wherein the protein subunit lacks a C-terminal transmembrane anchor and is expressed and secreted as a soluble protein from stably transformed insect cells.

20. An immunogenic composition comprising a recombinant subunit influenza vaccine comprising a recombinant influenza hemagglutinin head protein subunit, wherein the hemagglutinin head protein subunit lacks a C-terminal transmembrane anchor, lacks an N-terminal portion, and is expressed and secreted as a soluble protein from stably transformed insect cells.

21. An immunogenic composition comprising a recombinant subunit influenza vaccine comprising a recombinant influenza hemagglutinin ectodomain protein subunit, wherein the hemagglutinin ectodomain protein subunit lacks a C-terminal transmembrane anchor and is expressed and secreted as a soluble protein from stably transformed insect cells,
combined with a recombinant influenza matrix 1 protein subunit, wherein the matrix 1 protein subunit lacks a C-terminal transmembrane anchor, and is expressed and secreted as a soluble tetrameric protein from stably transformed insect cells.

22. An immunogenic composition comprising a recombinant subunit influenza vaccine comprising a recombinant influenza hemagglutinin head protein subunit, wherein the hemagglutinin head protein subunit lacks a C-terminal transmembrane anchor, lacks an N-terminal portion, and is expressed and secreted as a soluble protein from stably transformed insect cells,
combined with a recombinant influenza matrix 1 protein subunit, wherein the matrix 1 protein subunit lacks a C-terminal transmembrane anchor, and is expressed and secreted as a soluble tetrameric protein from stably transformed insect cells.

23. The immunogenic composition of claim 19, 20, 21, or 22, wherein the influenza virus is influenza A virus.

24. The immunogenic composition of claim 19, 20, 21, or 22, wherein the strain of influenza virus is selected from the group consisting of H5 and H3.

25. The immunogenic composition of claim 19, 20, 21, or 22, wherein the carboxy-terminal portion of the hemagglutinin protein subunit is truncated within 10% of the length of a nominal ectodomain.

26. The immunogenic composition of claim 19, 20, 21, or 22, wherein the stably transformed insect cells are Drosophila melanogaster S2 cells.

27. The immunogenic composition of claim 19, 20, 21, or 22, wherein the immunogenic composition further comprises one or more adjuvants.

28. The immunogenic composition of claim 19, 20, 21, or 22, wherein the immunogenic composition further comprises one or more adjuvants selected from the group consisting of saponin and alum.

29. The immunogenic composition of claim 19, 20, 21, or 22, wherein the immunogenic composition further comprises GPI-0100 adjuvant.

30. The immunogenic composition of claim 19, 20, 21, or 22, wherein the immunogenic composition further comprises a pharmaceutically acceptable excipient.

31. The immunogenic composition of claim 19, 20, 21, or 22, wherein the protein subunits are purified by immuno-affinity chromatography.

32. The immunogenic composition of claim 19, 20, 21, or 22, wherein the immunogenic composition is administered to a subject in a vaccine.

33. The immunogenic composition of claim 19 or 21, wherein the recombinant influenza hemagglutinin ectodomain protein subunit has an amino acid sequence selected from the group consisting of SEQ ID NO:1 and SEQ ID NO:2.

34. The immunogenic composition of claim 19 or 21, wherein the recombinant influenza hemagglutinin ectodomain protein subunit has an amino acid sequence with at least 95% sequence identity to an amino acid selected from the group consisting of SEQ ID NO:1 and SEQ ID NO:2.

35. The immunogenic composition of claim 19 or 21, wherein the recombinant influenza hemagglutinin ectodomain protein subunit has an amino acid sequence with at least 90% sequence identity to an amino acid selected from the group consisting of SEQ ID NO:1 and SEQ ID NO:2.

36. The immunogenic composition of claim 20 or 22, wherein the recombinant influenza hemagglutinin head protein subunit has an amino acid sequence selected from the group consisting of SEQ ID NO:3, SEQ ID NO:4, SEQ ID NO:5, and SEQ ID NO:6.

37. The immunogenic composition of claim 20 or 22, wherein the recombinant influenza hemagglutinin head protein subunit has an amino acid sequence with at least 95% sequence identity to an amino acid selected from the group consisting of SEQ ID NO:3, SEQ ID NO:4, SEQ ID NO:5, and SEQ ID NO:6.

38. The immunogenic composition of claim 20 or 22, wherein the recombinant influenza hemagglutinin head protein subunit has an amino acid sequence with at least 90% sequence identity to an amino acid selected from the group consisting of SEQ ID NO:3, SEQ ID NO:4, SEQ ID NO:5, and SEQ ID NO:6.

39. The immunogenic composition of claim 20 or 22, wherein the truncation points of the hemagglutinin head protein subunit are selected from the group consisting of N-terminal, C-terminal, and N-terminal and C-terminal, wherein the one or both terminal points can be varied up to 10% of the length of a nominal HA-head.

40. The immunogenic composition of claim 21 or 22, wherein the recombinant influenza matrix 1 protein subunit has the amino acid sequence of SEQ ID NO:7.

41. The immunogenic composition of claim 21 or 22, wherein the recombinant influenza matrix 1 protein subunit has an amino acid sequence with at least 95% sequence identity to SEQ ID NO:7.

42. The immunogenic composition of claim 21 or 22, wherein the recombinant influenza matrix 1 protein subunit has an amino acid sequence with at least 90% sequence identity to SEQ ID NO:7.


SEQUENCE LISTING (7 sequences)

SEQ ID NO: 1 - protein, 508 aa, Influenza A Virus -  A/Fujian/411/02 strain
GARSQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSACKRRSNKSFFSRLNWLTHLKYKYPALNVTMPNNEKFDKLYIWGVHHPGTDSDQISLYAQASGRITVSTKRSQQTVIPNIGSRPRVRDVSSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKG

SEQ ID NO: 2 - protein, 509 aa, Influenza A Virus -  A/Hong Kong/156/97 strain
GARSDQICIGYHANNSTEQVDTIMEKNVTVTHAQDILERTHNGKLCDLNGVKPLILRDCSVAGWLLGNPMCDEFINVPEWSYIVEKASPANDLCYPGNFNDYEELKHLLSRINHFEKIQIIPKSSWSNHDASSGVSSACPYLGRSSFFRNVVWLIKKNSAYPTIKRSYNNTNQEDLLVLWGIHHPNDAAEQTKLYQNPTTYISVGTSTLNQRLVPEIATRPKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNTPQRERRRKKRGLFGAIAGFIEGGWQGMVDGWYGYHHSNEQGSGYAADKESTQKAIDGVTNKVNSIINKMNTQFEAVGREFNNLERRIENLNKKMEDGFLDVWTYNAELLVLMENERTLDFHDSNVKNLYDKVRLQLRDNAKELGNGCFEFYHKCDNECMESVKNGTYDYPQYSEEARLNREEISG

SEQ ID NO: 3 - protein, 315 aa, Influenza A Virus
GARSAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSACKRRSNKSFFSRLNWLTHLKYKYPALNVTMPNNEKFDKLYIWGVHHPGTDSDQISLYAQASGRITVSTKRSQQTVIPNIGSRPRVRDVSSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTR

SEQ ID NO: 4 - protein, 285 aa, Influenza A Virus
GARSGGICDSPHQILDGENCTLIDALLGDPQCDGFQNKKWDLFVERSKAYSNCYPYDVPDYASLRSLVASSGTLEFNNESFNWTGVTQNGTSSACKRRSNKSFFSRLNWLTHLKYKYPALNVTMPNNEKFDKLYIWGVHHPGTDSDQISLYAQASGRITVSTKRSQQTVIPNIGSRPRVRDVSSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTR

SEQ ID NO: 5 - protein, 322 aa, Influenza A Virus
GARSANNSTEQVDTIMEKNVTVTHAQDILERTHNGKLCDLNGVKPLILRDCSVAGWLLGNPMCDEFINVPEWSYIVEKASPANDLCYPGNFNDYEELKHLLSRINHFEKIQIIPKSSWSNHDASSGVSSACPYLGRSSFFRNVVWLIKKNSAYPTIKRSYNNTNQEDLLVLWGIHHPNDAAEQTKLYQNPTTYISVGTSTLNQRLVPEIATRPKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNTPQRERR

SEQ ID NO: 6 - protein, 292 aa, Influenza A Virus
GARSGKLCDLNGVKPLILRDCSVAGWLLGNPMCDEFINVPEWSYIVEKASPANDLCYPGNFNDYEELKHLLSRINHFEKIQIIPKSSWSNHDASSGVSSACPYLGRSSFFRNVVWLIKKNSAYPTIKRSYNNTNQEDLLVLWGIHHPNDAAEQTKLYQNPTTYISVGTSTLNQRLVPEIATRPKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNTPQRERR

SEQ ID NO: 7 - protein, 256 aa, Influenza A Virus -  A/Hong Kong/156/97 strain
GARSMSLLTEVETYVLSIIPSGPLKAEIAQRLEDVFAGKNTDLEALMEWLKTRPILSPLTKGILGFVFTLTVPSERGLQRRRFVQNALNGNGDPNNMDRAVKLYKKLKREMTFHGAKEVALSYSTGALASCMGLIYNRMGTVTTEVALGLVCATCEQIADAQHRSHRQMATTTNPLIRHENRMVLASTTAKAMEQMAGSSEQAAEAMEVASQARQMVQAMRTIGTHPSSSAGLKDDLIENLQAYQKRMGVQMQRFK